(19) United States
(12) Patent Application Publication
(10) Pub. No.: US 2024/0200140 A1
(43) Pub. Date: Jun. 20, 2024

(54) COMPOSITIONS AND METHODS FOR DIAGNOSING AND TREATING CANCER

(71) Applicant: The Regents of the University of California, Oakland, CA (US)

(72) Inventor: Daniel H. KIM, Santa Cruz, CA (US)

(73) Assignee: The Regents of the University of California, Oakland, CA (US)

(21) Appl. No.: 17/768,786

(22) PCT Filed: Oct. 19, 2020
(86) PCT No.: PCT/US2020/056316

§ 371 Date: Dec. 28, 2023

Related U.S. Application Data
(60) Provisional application No. 62/923,127, filed on Oct. 18, 2019.

(51) Int. Cl. C12Q 1/6886; A61K 45/06; C12Q 1/686

(52) U.S. Cl. CPC C12Q 1/6886; A61K 45/06; C12Q 1/686; C12Q 2600/106; C12Q 2600/158

(57) ABSTRACT

The disclosure provides methods for diagnosing and/or treating cancer in a subject by measuring the expression level of one or more genes listed in Tables 1-3 in a biological sample from the subject.

### BACKGROUND

Oncogenic KRAS is a potent initiator of tumorigenesis, yet its nascent effects on the noncoding genome are incompletely understood.

### SUMMARY

In one aspect, the disclosure features a method for diagnosing and/or treating cancer in a subject, the method comprising: analyzing the expression level of one or more genes in Tables 1-3 in a biological sample from the subject in conjunction with a corresponding reference level for the gene in a control sample from a control subject, wherein a differential expression level of the one or more genes in the biological sample from the subject compared to the corresponding reference level for the gene in the control sample from the control subject indicates that the subject has cancer.

In some embodiments, the method further comprises, prior to analyzing, measuring the expression level of the one or more genes in Tables 1-3 and the expression level of the corresponding reference level for the gene in the control sample. In some embodiments, the method further comprises, after analyzing, administering to the subject one or more anticancer agents. In certain embodiments, the anticancer agent is an inhibitor of a K-ras gene. In other embodiments, the anticancer agent is an inhibitor of the gene that is identified to have the differential expression level compared to the corresponding reference level for the gene in the control sample.

In some embodiments, the cancer comprises a KRAS mutation. The KRAS mutation can be in a tissue of the subject, such as lung tissue. In certain embodiments, the cancer is lung cancer, such as lung adenocarcinoma.

In some embodiments, the method comprises analyzing the expression level of a gene involved in the interferon (IFN) alpha or gamma response. In certain embodiments, an increase in the expression level of the gene involved in the IFN alpha or gamma response relative to a corresponding reference level for the gene in the control sample from the control subject indicates that the subject has cancer.

In some embodiments, the method comprises analyzing the expression level of a gene encoding a pattern recognition receptor (PRR). In certain embodiments, an increase in the expression level of the gene encoding the PRR relative to a corresponding reference level for the gene in the control sample from the control subject indicates that the subject has cancer. In some embodiments, the method comprises analyzing the expression level of a gene encoding cytosolic RNA sensor RIG-I or MDA5. In certain embodiments, an increase in the expression level of the gene encoding the cytosolic RNA sensor RIG-I or MDA5 relative to a corresponding reference level for the gene in the control sample from the control subject indicates that the subject has cancer.

In some embodiments, the method comprises analyzing the expression level of a gene encoding a KRAB zinc-finger (KZNF) protein. In certain embodiments, a decrease in the expression level of the gene encoding the KZNF protein relative to a corresponding reference level for the gene in the control sample from the control subject indicates that the subject has cancer.

In some embodiments, measuring the expression level of the one or more genes comprises performing polymerase chain reaction (PCR), reverse transcriptase polymerase chain reaction (RT-PCR), single-cell RNA-sequencing, microarray analysis, a Northern blot, serial analysis of gene expression (SAGE), immunoassay, hybridization capture, cDNA sequencing, direct RNA sequencing, nanopore sequencing, and/or mass spectrometry. Specifically, when PCR is used to measure the expression level, at least one set of oligonucleotide primers comprising a forward primer and a reverse primer capable of amplifying a polynucleotide sequence of the gene can be used.

In some embodiments, the biological sample is a blood sample, a urine sample, or a tissue sample (e.g., a blood sample). In some embodiments, the subject suspected of having cancer or in need of treatment is a mammal (e.g., a human).

In another aspect, the disclosure also features a biomarker panel comprising two or more genes listed in Tables 1-3.

### Definitions

As used herein, the term “KRAS mutation” refers to a genetic mutation in the K-ras gene, which acts as an on-off switch in cell signaling and controls cell proliferation.

As used herein, the term “long noncoding RNA” or “lncRNA” refers to RNA polynucleotides that are not translated into proteins. Long ncRNAs may vary in length from several hundred bases to tens of kilo bases (e.g., at least 100, 200, 300, 400, 500, 600, 700, 800, 900, 1000, 1100, 1200, 1300, 1400, 1500, 1600, 1700, 1800, 1900, or 2000 bases) and may be located separately from protein coding genes, or reside near or within protein coding genes.

As used herein, the term “polynucleotide” refers to an oligonucleotide, or nucleotide, and fragments or portions thereof, and to DNA or RNA of genomic or synthetic origin, which may be single- or double-stranded, and represent the sense or anti-sense strand. A single polynucleotide is translated into a single polypeptide.

As used herein, the terms “peptide” and “polypeptide” are used interchangeably and describe a single polymer in which the monomers are amino acid residues which are joined together through amide bonds. A polypeptide is intended to encompass any amino acid sequence, either naturally occurring, recombinant, or synthetically produced.

As used herein, the term “substantial identity” or “substantially identical,” used in the context of nucleic acids or polypeptides, refers to a sequence that has at least 50% sequence identity with a reference sequence. Alternatively, percent identity can be any integer from 50% to 100%. In some embodiments, a sequence is substantially identical to a reference sequence if the sequence has at least 50%, 55%, 60%, 65%, 70%, 75%, 80%, 85%, 90%, 91%, 92%, 93%, 94%, 95%, 96%, 97%, 98%, or 99% sequence identity to the reference sequence as determined using, e.g., BLAST.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A comparison window includes reference to a segment of any one of the number of contiguous positions, e.g., a segment of at least 10 residues. In some embodiments, the comparison window has from 10 to 600 residues, e.g., about 10 to about 30 residues, about 10 to about 20 residues, about 50 to about 200 residues, or about 100 to about 150 residues, in which a sequence may be compared to a reference sequence of the same number of contiguous positions after the two sequences are optimally aligned.

Algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0)). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, an amino acid sequence is considered similar to a reference sequence if the smallest sum probability in a comparison of the test amino acid sequence to the reference amino acid sequence is less than about 0.01, more preferably less than about 10⁻⁵, and most preferably less than about 10⁻²⁰.

### BRIEF DESCRIPTION OF THE DRAWINGS

FIGS. 1A-ID. Tissue-specific transcriptome reprogramming by mutant KRAS. (A) Chromosome-level distribution of differentially expressed RNAs in mutant KRAS lung epithelial cells (AALE). Shown are the two most abundant biotypes from RNA-seq data. (B) Gene set enrichment analysis (GSEA) pathways sorted by normalized enrichment score (NES) in mutant KRAS lung epithelial cells. (C) Chromosome-level distribution of differentially expressed RNAs in mutant KRAS kidney cells (HA1E). (D) GSEA pathways sorted by NES in mutant KRAS kidney cells.

FIGS. 2A-2E. Mutant KRAS activates IFN-related genes and transposable elements. Differentially expressed interferon-stimulated genes in (A) mutant KRAS lung epithelial cells and (B) mutant KRAS kidney cells. (C) Cell viability in mutant KRAS lung epithelial cells transfected with indicated small interfering RNAs. Differentially expressed transposable elements in (D) mutant KRAS lung epithelial cells and (E) mutant KRAS kidney cells.

FIGS. 3A-3F. Coordinate regulation of IFN-related genes and transposable elements. Uniform manifold approximation and projection (UMAP) visualization of single-cell RNA-seq (scRNA-seq) data from mutant KRAS lung epithelial cells showing (A) clustering and expression of (B) IFN beta and (C) RIG-I/MDA5 metagenes. (D-F) Correlations between transposable elements and IFN-related metagenes in scRNA-seq clusters.

FIGS. 4A-4G. Broad suppression of KRAB zinc finger proteins in lung cancer cells. Differentially expressed zinc finger proteins in (A) mutant KRAS lung epithelial cells and (B) mutant KRAS kidney cells. ChIP-seq data from indicated zinc finger proteins showing binding to the consensus sequences of (C) THE1D, (D) MER20, and (E) L1MC4a. (F) Significantly repressed zinc finger proteins in mutant KRAS lung adenocarcinomas compared to matched normal lung samples and (G) their corresponding expression levels in kidney cancers compared to matched normal kidney samples.

FIGS. 5A-5D. Transcriptome reprogramming by mutant KRAS. (A) Chromosome-level distribution of differentially expressed RNAs in mutant lung epithelial cells. (B) Proportion of exons that overlap a transposable element (TE) for all genes detected and differentially expressed in mutant lung epithelial cells, separated by biotype. (C) Chromosome-level distribution of differentially expressed RNAs in mutant kidney cells. (D) Proportion of exons that overlap a transposable element (TE) for all genes detected and differentially expressed in mutant kidney cells, separated by biotype.

FIGS. 6A-6D. Interferon-stimulated gene expression heterogeneity in transformed cells. Uniform manifold approximation and projection (UMAP) visualization of single-cell RNA-seq data from mutant KRAS lung epithelial cells showing expression of indicated metagenes.

### DETAILED DESCRIPTION OF THE EMBODIMENTS

### I. Introduction

Most of the human genome is noncoding and transcribed into RNA (1, 2), but how the noncoding transcriptome contributes to cancer formation is poorly understood. About half of the human genome is comprised of transposable elements (TE) (3), whose expression patterns are often altered in cancer (4). Additionally, TEs contribute substantially to the noncoding transcriptome and are present in the exonic sequences of thousands of long noncoding RNAs (lncRNAs) and other classes of regulatory RNAs (5). Noncoding RNA networks become disrupted in cancer (6, 7) and epigenetic reprogramming, where early activation of RAS signaling leads to coordinate activation of noncoding RNAs in single cells (8). While RAS genes are among the most frequently mutated oncogenes in cancer (9), the extent to which RAS regulates the noncoding transcriptome during cellular transformation remains unknown.

To determine the landscape of noncoding RNAs affected by oncogenic RAS signaling, we performed RNA sequencing (RNA-seq) on human lung epithelial cells (AALE) that undergo malignant transformation upon introduction of mutant KRAS (10). We compared the transcriptomes of AALE cells transduced with control vector to AALEs that were transformed by mutant KRAS and analyzed the distribution of differentially expressed transcripts across the genome.

### II. Transcriptome Affected by Oncogenic RAS Signaling

We analyzed the transcriptomes of human lung and kidney cells transformed with mutant KRAS to define the landscape of RAS-regulated noncoding RNAs. We found that oncogenic RAS upregulates noncoding transcripts throughout the genome, many of which arise from transposable elements. These repetitive sequences are preferential targets of KRAB zinc-finger proteins, which are broadly downregulated in mutant KRAS cells and lung adenocarcinomas. Moreover, KRAS-mediated reprogramming of repetitive noncoding RNA induces an interferon response that contributes to cellular transformation. The results reveal the extent to which mutant KRAS remodels the noncoding transcriptome, expanding the scope of genomic elements regulated by this fundamental signaling pathway.

Tables 1-3 below list genes whose expression levels are found to be altered by mutant KRAS. The disclosure relates to the genes listed in Tables 1-3 and their diagnostic and therapeutic uses for cancer (e.g., lung cancer). In some embodiments, one or more genes disclosed herein have a differential expression induced by mutant KRAS. As described herein, dynamic changes in the transcriptome were observed in AALE cells transformed by mutant KRAS. Furthermore, the expression of some genes were found to be specifically induced by mutant KRAS in cells from a given tissue type. These results reveal that KRAS-induced genetic signatures are tissue-specific. In some embodiments of the compositions and methods described herein, a plurality of the genes listed in Tables 1-3 can be used to identify KRAS mutations in a tissue specific manner, leading to potentially identifying and diagnosing various types of cancer in their early stages and applying appropriate treatments.

TABLE 1
Intron Biomarkers

| | | | | | | | | | p- | | |
|---|---|---|---|---|---|---|---|---|---|---|---|
| enst | chromosome | start.position | end.position | strand | transcript.id | gene | len | log2FoldChange | value | biotype | genome |
| ENST000005 | 14 | 100361703 | 100375473 | − | WARS- | WARS | 582 | 3.293972806 | 0.000854 | retained- | hg38 |
| 57094.5 | | | | | 237 | | | | | intron | |
| ENST000005 | 5 | 146261383 | 146263519 | + | RBM27- | RBM27 | 453 | 3.137712098 | 0.004569 | retained- | hg38 |
| 08019.1 | | | | | 202 | | | | | intron | |
| ENST000004 | 1 | 148290889 | 148296776 | − | LINC01138- | LINC01138 | 1143 | 1.934244609 | 6.08E−06 | retained- | hg38 |
| 45201.2 | | | | | 203 | | | | | intron | |
| ENST000005 | 5 | 73058421 | 73077440 | + | FCHO2- | FCHO2 | 553 | 1.927973168 | 0.00896 | retained- | hg38 |
| 08431.1 | | | | | 205 | | | | | intron | |
| ENST000005 | 1 | 169303100 | 169367782 | − | NME7- | NME7 | 557 | 1.843357221 | 0.024478 | retained- | hg38 |
| 27460.1 | | | | | 212 | | | | | intron | |
| ENST000004 | 17 | 30477417 | 30490350 | + | GOSR1- | GOSR1 | 584 | 1.841994189 | 0.025001 | retained- | hg38 |
| 67635.6 | | | | | 206 | | | | | intron | |
| ENST000004 | 7 | 99498585 | 99499704 | − | ZNF394- | ZNF394 | 325 | 1.809825585 | 0.011592 | retained- | hg38 |
| 64401.1 | | | | | 205 | | | | | intron | |
| ENST000005 | 5 | 34914477 | 34915504 | − | RAD1- | RAD1 | 574 | 1.796670164 | 0.041416 | retained- | hg38 |
| 06311.1 | | | | | 204 | | | | | intron | |
| ENST000004 | 2 | 110642114 | 110678028 | − | BUB1-207 | BUB1 | 2501 | 1.784091007 | 0.003847 | retained- | hg38 |
| 66333.5 | | | | | | | | | | intron | |
| ENST000005 | 4 | 150265026 | 150315634 | − | LRBA- | LRBA | 1636 | 1.77019687 | 0.007722 | retained- | hg38 |
| 10157.1 | | | | | 208 | | | | | intron | |
| ENST000006 | 1 | 155191863 | 155192909 | − | MUC1- | MUC1 | 572 | 1.730092884 | 0.009695 | retained- | hg38 |
| 20770.1 | | | | | 229 | | | | | intron | |
| ENST000004 | 1 | 114720216 | 114726348 | − | CSDE1- | CSDE1 | 873 | 1.601512932 | 0.015137 | retained- | hg38 |
| 83030.1 | | | | | 206 | | | | | intron | |
| ENST000005 | 17 | 4968139 | 4969081 | − | CAMTA2- | CAMTA2 | 856 | 1.556293725 | 4.97E−05 | retained- | hg38 |
| 72192.1 | | | | | 206 | | | | | intron | |
| ENST000005 | 17 | 4734583 | 4738539 | − | CXCL16- | CXCL16 | 619 | 1.459348853 | 0.049021 | retained- | hg38 |
| 75168.1 | | | | | 204 | | | | | intron | |
| ENST000004 | 9 | 128120752 | 128125253 | − | PTGES2- | PTGES2 | 1514 | 1.358396555 | 0.036463 | retained- | hg38 |
| 93205.5 | | | | | 211 | | | | | intron | |
| ENST000005 | 1 | 1629106 | 1630603 | + | MIB2-227 | MIB2 | 584 | 1.3503614 | 0.001572 | retained- | hg38 |
| 11910.1 | | | | | | | | | | intron | |
| ENST000005 | 1 | 1615514 | 1630604 | + | MIB2-226 | MIB2 | 3326 | 1.292032367 | 0.030268 | retained- | hg38 |
| 11502.5 | | | | | | | | | | intron | |
| ENST000004 | 22 | 40408388 | 40410047 | + | SGSM3- | SGSM3 | 1029 | 1.230036111 | 0.017716 | retained- | hg38 |
| 69719.5 | | | | | 207 | | | | | intron | |
| ENST000004 | 3 | 184319625 | 184321534 | + | EIF4G1- | EIF4G1 | 590 | 1.228742301 | 0.044799 | retained- | hg38 |
| 84862.5 | | | | | 236 | | | | | intron | |
| ENST000005 | 9 | 22005203 | 22006271 | − | CDKN2B- | CDKN2B | 1069 | 1.11814584 | 1.15E−06 | retained- | hg38 |
| 79591.1 | | | | | 203 | | | | | intron | |
| ENST000004 | 15 | 99136406 | 99221864 | − | TTC23- | TTC23 | 2952 | 1.069833641 | 0.032316 | retained- | hg38 |
| 94567.1 | | | | | 211 | | | | | intron | |
| ENST000004 | 11 | 1834310 | 1837521 | + | SYT8-209 | SYT8 | 1956 | 1.063723564 | 0.000806 | retained- | hg38 |
| 79089.5 | | | | | | | | | | intron | |
| ENST000004 | 21 | 43053191 | 43068404 | − | CBS-209 | CBS | 2656 | 1.025934645 | 0.020483 | retained- | hg38 |
| 61686.5 | | | | | | | | | | intron | |
| ENST000003 | 20 | 63570139 | 63574239 | − | HELZ2- | HELZ2 | 1827 | 1.025633626 | 0.000139 | retained- | hg38 |
| 70082.1 | | | | | 201 | | | | | intron | |
| ENST000005 | 5 | 134388138 | 134390605 | + | UBE2B- | UBE2B | 657 | 1.011036613 | 0.013832 | retained- | hg38 |
| 03080.1 | | | | | 203 | | | | | intron | |
| ENST000005 | 11 | 57741699 | 57743514 | + | SELENOH- | SELENOH | 1039 | 1.005915525 | 0.041306 | retained- | hg38 |
| 34386.2 | | | | | 205 | | | | | intron | |
| ENST000005 | 5 | 178208654 | 178230320 | − | PHYKPL- | PHYKPL | 814 | 1.005802616 | 0.034591 | retained- | hg38 |
| 10991.5 | | | | | 216 | | | | | intron | |
| ENST000004 | 6 | 27251213 | 27255908 | + | PRSS16- | PRSS16 | 1002 | 1.000739737 | 0.031397 | retained- | hg38 |
| 92575.5 | | | | | 219 | | | | | intron | |

TABLE 2
Protein Coding Biomakers

| | chromo- | | | | | | | | p- | | |
|---|---|---|---|---|---|---|---|---|---|---|---|
| enst | some | start.position | end.position | strand | transcript.id | gene | len | log2FoldChange | value | biotype | genome |
| ENST00000361 | 1 | 27666061 | 27672218 | − | IFI6-202 | IFI6 | 841 | 3.75726403 | 9.48E−07 | protein- | hg38 |
| 157.10 | | | | | | | | | | coding | |
| ENST00000649 | 16 | 86566829 | 86569728 | + | FOXC2-202 | FOXC2 | 2900 | 3.738134499 | 2.35E−07 | protein- | hg38 |
| 859.1 | | | | | | | | | | coding | |
| ENST00000256 | 12 | 25209431 | 25250803 | − | KRAS-201 | KRAS | 1119 | 3.688899635 | 3.8E−05 | protein- | hg38 |
| 078.8 | | | | | | | | | | coding | |
| ENST00000261 | 12 | 20815672 | 20916911 | + | SLCO1B3- | SLCO1B3 | 2840 | 3.509148933 | 1.09E−06 | protein- | hg38 |
| 196.6 | | | | | 201 | | | | | coding | |
| ENST00000524 | 6 | 99545168 | 99568227 | − | CCNC-218 | CCNC | 759 | 3.428836274 | 0.0023 | protein- | hg38 |
| 049.5 | | | | | | | | | | coding | |
| ENST00000275 | X | 34627064 | 34657288 | − | TMEM47- | TMEM47 | 4054 | 3.345762035 | 1.43E−08 | protein- | hg38 |
| 954.3 | | | | | 201 | | | | | coding | |
| ENST00000371 | 10 | 89392546 | 89403988 | + | IFIT1-201 | IFIT1 | 1880 | 3.296687078 | 1.29E−09 | protein- | hg38 |
| 804.3 | | | | | | | | | | coding | |
| ENST00000320 | 16 | 86567251 | 86569728 | + | FOXC2-201 | FOXC2 | 2478 | 3.164092038 | 0.00395 | protein- | hg38 |
| 354.5 | | | | | | | | | | coding | |
| ENST00000327 | 12 | 52285913 | 52291534 | − | KRT81-201 | KRT81 | 1929 | 3.12422172 | 2.5E−08 | protein- | hg38 |
| 741.9 | | | | | | | | | | coding | |
| ENST00000371 | 10 | 89301955 | 89309276 | + | IFIT2-201 | IFIT2 | 3489 | 3.107028696 | 1.64E−07 | protein- | hg38 |
| 826.3 | | | | | | | | | | coding | |
| ENST00000341 | 11 | 1834804 | 1837521 | + | SYT8-201 | SYT8 | 1556 | 3.057400577 | 0.00045 | protein- | hg38 |
| 958.3 | | | | | | | | | | coding | |
| ENST00000398 | 21 | 41426167 | 41459214 | + | MX1-202 | MX1 | 2850 | 2.965414141 | 0.000954 | protein- | hg38 |
| 598.7 | | | | | | | | | | coding | |
| ENST00000257 | 12 | 121019111 | 121039242 | − | OASL-201 | OASL | 3266 | 2.963031748 | 1.16E−07 | protein- | hg38 |
| 570.9 | | | | | | | | | | coding | |
| ENST00000370 | 1 | 78649831 | 78664078 | + | IFI44-201 | IFI44 | 1687 | 2.94788013 | 1.94E−07 | protein- | hg38 |
| 747.8 | | | | | | | | | | coding | |
| ENST00000508 | 5 | 94708549 | 95081645 | − | MCTP1- | MCTP1 | 2214 | 2.832339587 | 0.00874 | protein- | hg38 |
| 509.5 | | | | | 209 | | | | | coding | |
| ENST00000621 | 14 | 94110749 | 94116695 | + | IFI27-215 | IFI27 | 644 | 2.826852134 | 0.000734 | protein- | hg38 |
| 160.4 | | | | | | | | | | coding | |
| ENST00000649 | 1 | 1013497 | 1014540 | + | ISG15-204 | ISG15 | 637 | 2.786256252 | 4.44E−10 | protein- | hg38 |
| 529.1 | | | | | | | | | | coding | |
| ENST00000339 | 12 | 121020557 | 121039156 | − | OASL-202 | OASL | 1492 | 2.774555087 | 0.00099 | protein- | hg38 |
| 275.9 | | | | | | | | | | coding | |
| ENST00000424 | 6 | 31647604 | 31652667 | − | BAG6-240 | BAG6 | 1056 | 2.772644748 | 0.006213 | protein- | hg38 |
| 480.5 | | | | | | | | | | coding | |
| ENST00000371 | 10 | 89327894 | 89340971 | + | IFIT3-202 | IFIT3 | 2496 | 2.747204085 | 0.006352 | protein- | hg38 |
| 818.8 | | | | | | | | | | coding | |
| ENST00000362 | 1 | 27666066 | 27672198 | − | IFI6-203 | IFI6 | 828 | 2.722538533 | 0.002499 | protein- | hg38 |
| 020.4 | | | | | | | | | | coding | |
| ENST00000566 | 16 | 1533573 | 1555580 | + | TMEM204- | TMEM204 | 1938 | 2.69851486 | 7.25E−05 | protein- | hg38 |
| 264.1 | | | | | 202 | | | | | coding | |
| ENST00000367 | 1 | 196651878 | 196747504 | + | CFH-202 | CFH | 4127 | 2.646360432 | 5.511-08 | protein- | hg38 |
| 429.8 | | | | | | | | | | coding | |
| ENST00000371 | 1 | 47023568 | 47050751 | + | CYP4X1- | CYP4X1 | 2357 | 2.642345543 | 6.06E−06 | protein- | hg38 |
| 901.3 | | | | | 201 | | | | | coding | |
| ENST00000255 | 11 | 63536821 | 63546462 | + | RARRES3- | RARRES3 | 749 | 2.588686336 | 0.000117 | protein- | hg38 |
| 688.7 | | | | | 201 | | | | | coding | |
| ENST00000370 | 1 | 85652808 | 85708418 | − | ZNHIT6 | ZNHIT6 | 2797 | 2.571831117 | 1.01E−09 | protein- | hg38 |
| 574.3 | | | | | 201 | | | | | coding | |
| ENST00000611 | 10 | 89302046 | 89308919 | + | IFIT2-202 | IFIT2 | 3038 | 2.526327895 | 8.19E−07 | protein- | hg38 |
| 722.1 | | | | | | | | | | coding | |
| ENST00000264 | 4 | 88457117 | 88506163 | + | HERC5-201 | HERC5 | 3513 | 2.503694262 | 1.84E−08 | protein- | hg38 |
| 350.7 | | | | | | | | | | coding | |
| ENST00000349 | 2 | 227325276 | 227357812 | + | MFF-203 | MFF | 1716 | 2.406702952 | 0.00163 | protein- | hg38 |
| 901.11 | | | | | | | | | | coding | |
| ENST00000645 | 1 | 6424776 | 6460944 | + | ESPN-218 | ESPN | 3543 | 2.394876462 | 0.000459 | protein- | hg38 |
| 284.1 | | | | | | | | | | coding | |
| ENST00000429 | 5 | 94706579 | 95081645 | − | MCTP1- | MCTP1 | 3159 | 2.370601632 | 6.59E−06 | protein- | hg38 |
| 576.6 | | | | | 202 | | | | | coding | |
| ENST00000339 | 1 | 1512530 | 1534685 | + | ATAD3A- | ATAD3A | 2330 | 2.312568468 | 0.003414 | protein- | hg38 |
| 113.8 | | | | | 201 | | | | | coding | |
| ENST00000360 | 16 | 55802853 | 55833158 | − | CES1-201 | CES1 | 2006 | 2.312212075 | 8.64E−05 | protein- | hg38 |
| 526.7 | | | | | | | | | | coding | |
| ENST00000618 | 14 | 94110747 | 94116447 | + | IFI27-211 | IFI27 | 364 | 2.305518338 | 0.001392 | protein- | hg38 |
| 200.4 | | | | | | | | | | coding | |
| ENST00000514 | 5 | 33440739 | 33453346 | + | TARS-214 | TARS | 466 | 2.252059871 | 0.025226 | protein- | hg38 |
| 259.5 | | | | | | | | | | coding | |
| ENST00000371 | 10 | 89332484 | 89340971 | + | IFIT3-201 | IFIT3 | 2455 | 2.193840753 | 0.006943 | protein- | hg38 |
| 811.4 | | | | | | | | | | coding | |
| ENST00000555 | 14 | 75279643 | 75281684 | + | FOS-208 | FOS | 1496 | 2.157727932 | 1.96E−05 | protein- | hg38 |
| 686.1 | | | | | | | | | | coding | |
| ENST00000649 | 2 | 162267079 | 162318652 | − | IFIH1-207 | IFIH1 | 3544 | 2.155055238 | 0.00378 | protein- | hg38 |
| 979.1 | | | | | | | | | | coding | |
| ENST00000349 | 4 | 102797264 | 102827849 | − | UBE2D3- | UBE2D3 | 838 | 2.112175875 | 0.031604 | protein- | hg38 |
| 311.12 | | | | | 204 | | | | | coding | |
| ENST00000368 | 6 | 122610232 | 122725892 | + | PKIB-205 | PKIB | 1398 | 2.069071583 | 0.001777 | protein- | hg38 |
| 452.6 | | | | | | | | | | coding | |
| ENST00000603 | 17 | 35871491 | 35880508 | − | CCL5-201 | CCL5 | 1365 | 2.067195368 | 0.002275 | protein- | hg38 |
| 197.5 | | | | | | | | | | coding | |
| ENST00000265 | 11 | 68754889 | 68841916 | − | CPT1A-201 | CPT1A | 5232 | 2.02881839 | 1.61E−07 | protein- | hg38 |
| 641.9 | | | | | | | | | | coding | |
| ENST00000396 | 14 | 24161053 | 24166565 | + | IRF9-202 | IRF9 | 1838 | 2.020578106 | 0.000772 | protein- | hg38 |
| 864.7 | | | | | | | | | | coding | |
| ENST00000635 | 15 | 63153853 | 63157477 | − | RPS27L- | RPS27L | 693 | 2.011919702 | 0.005308 | protein- | hg38 |
| 699.1 | | | | | 208 | | | | | coding | |
| ENST00000620 | 14 | 94110815 | 94116698 | + | IFI27-213 | IFI27 | 719 | 2.010270782 | 0.00134 | protein- | hg38 |
| 066.1 | | | | | | | | | | coding | |
| ENST00000402 | 4 | 165378942 | 165498320 | + | CPE−201 | CPE | 2421 | 2.006566553 | 2.7E−09 | protein- | hg38 |
| 744.8 | | | | | | | | | | coding | |
| ENST00000555 | 14 | 75280193 | 75281587 | + | FOS-206 | FOS | 1280 | 1.981480408 | 2.5E−06 | protein- | hg38 |
| 347.1 | | | | | | | | | | coding | |
| ENST00000618 | 14 | 94110734 | 94116690 | + | IFI27-212 | IFI27 | 505 | 1.952488836 | 0.005933 | protein- | hg38 |
| 863.1 | | | | | | | | | | coding | |
| ENST00000644 | 4 | 153684278 | 153705378 | + | TLR2-206 | TLR2 | 2716 | 1.939352526 | 0.033911 | protein- | hg38 |
| 308.1 | | | | | | | | | | coding | |
| ENST00000577 | 17 | 47650358 | 47658641 | + | KPNB1-204 | KPNB1 | 605 | 1.926684917 | 0.037346 | protein- | hg38 |
| 875.5 | | | | | | | | | | coding | |
| ENST00000395 | 16 | 28537537 | 28539008 | − | NUPR1-202 | NUPR1 | 550 | 1.923381458 | 2.37E−08 | protein- | hg38 |
| 641.2 | | | | | | | | | | coding | |
| ENST00000264 | 4 | 23792021 | 23890077 | − | PPARGC1A- | PPARG | 6318 | 1.92147295 | 3.09E−05 | protein- | hg38 |
| 867.6 | | | | | 201 | CIA | | | | coding | |
| ENST00000397 | 1 | 41027200 | 41152674 | − | SCMH1- | SCMHI | 2977 | 1.90247111 | 0.005399 | protein- | hg38 |
| 174.6 | | | | | 209 | | | | | coding | |
| ENST00000593 | 19 | 46716165 | 46717112 | − | PRKD2-203 | PRKD2 | 669 | 1.893650203 | 0.029658 | protein- | hg38 |
| 363.1 | | | | | | | | | | coding | |
| ENST00000264 | 4 | 88378863 | 88443111 | + | HERC6-201 | HERC6 | 3779 | 1.886218536 | 5.67E−07 | protein- | hg38 |
| 346.11 | | | | | | | | | | coding | |
| ENST00000554 | 14 | 75278826 | 75280374 | + | FOS-204 | FOS | 796 | 1.883078887 | 0.000123 | protein- | hg38 |
| 617.1 | | | | | | | | | | coding | |
| ENST00000539 | 11 | 68757613 | 68815503 | − | CPT1A-205 | CPT1A | 2382 | 1.877494669 | 0.014155 | protein- | hg38 |
| 743.5 | | | | | | | | | | coding | |
| ENST00000382 | 2 | 6877665 | 6898239 | + | RSAD2-201 | RSAD2 | 3519 | 1.866944846 | 4.97E−06 | protein- | hg38 |
| 040.3 | | | | | | | | | | coding | |
| ENST00000560 | 15 | 88636153 | 88655621 | + | ISG20-210 | ISG20 | 800 | 1.8559211 | 2.37E−08 | protein- | hg38 |
| 741.5 | | | | | | | | | | coding | |
| ENST00000439 | 18 | 59430939 | 59697423 | − | CCBE1-202 | CCBE1 | 6271 | 1.848695376 | 0.001875 | protein- | hg38 |
| 986.9 | | | | | | | | | | coding | |
| ENST00000611 | 1 | 155185826 | 155192915 | − | MUC1-223 | MUC1 | 4170 | 1.830573122 | 1.47E−05 | protein- | hg38 |
| 571.4 | | | | | | | | | | coding | |
| ENST00000449 | 15 | 72199029 | 72231386 | − | PKM-204 | PKM | 2526 | 1.82190888 | 0.032014 | protein- | hg38 |
| 901.6 | | | | | | | | | | coding | |
| ENST00000225 | 17 | 19737984 | 19748433 | − | ALDH3A1- | ALDH3A1 | 1779 | 1.818651351 | 0.011605 | protein- | hg38 |
| 740.10 | | | | | 201 | | | | | coding | |
| ENST00000238 | 1 | 236523873 | 236544815 | + | LGALS8- | LGALS8 | 819 | 1.812904519 | 0.007013 | protein- | hg38 |
| 181.11 | | | | | 201 | | | | | coding | |
| ENST00000361 | 16 | 55803049 | 55833186 | − | CES1-202 | CES1 | 1835 | 1.805021916 | 0.000405 | protein- | hg38 |
| 503.8 | | | | | | | | | | coding | |
| ENST00000515 | 5 | 94705100 | 95284575 | − | MCTP1- | MCTP1 | 5396 | 1.798059507 | 9.07E−05 | protein- | hg38 |
| 393.5 | | | | | 218 | | | | | coding | |
| ENST00000431 | 1 | 85649423 | 85708433 | − | ZNHIT6- | ZNHIT6 | 6080 | 1.794909874 | 2.51E−07 | protein- | hg38 |
| 532.6 | | | | | 202 | | | | | coding | |
| ENST00000511 | 4 | 182243429 | 182803024 | + | TENM3- | TENM3 | 10896 | 1.793430257 | 3.73E−08 | protein- | hg38 |
| 685.5 | | | | | 204 | | | | | coding | |
| ENST00000233 | 2 | 187464261 | 187554492 | − | TFPI-201 | TFPI | 3885 | 1.793373089 | 0.000747 | protein- | hg38 |
| 156.8 | | | | | | | | | | coding | |
| ENST00000393 | 4 | 168216293 | 168318807 | − | DDX60-201 | DDX60 | 6071 | 1.791330976 | 1.8E−08 | protein- | hg38 |
| 743.7 | | | | | | | | | | coding | |
| ENST00000393 | 14 | 94612377 | 94624052 | + | SERPINA3- | SERPINA3 | 1589 | 1.785023983 | 0.022336 | protein- | hg38 |
| 078.4 | | | | | 201 | | | | | coding | |
| ENST00000553 | 14 | 30622329 | 30650626 | + | SCFD1-210 | SCFD1 | 490 | 1.78429222 | 0.032027 | protein- | hg38 |
| 693.5 | | | | | | | | | | coding | |
| ENST00000642 | 4 | 153684265 | 153705702 | + | TLR2-202 | TLR2 | 2979 | 1.781685949 | 0.021563 | protein- | hg38 |
| 580.1 | | | | | | | | | | coding | |
| ENST00000525 | 11 | 105026209 | 105035149 | − | CASP1-204 | CASP1 | 1237 | 1.78165249 | 0.001462 | protein- | hg38 |
| 825.5 | | | | | | | | | | coding | |
| ENST00000381 | 11 | 1834590 | 1837521 | + | SYT8-203 | SYT8 | 1291 | 1.778062994 | 0.02412 | protein- | hg38 |
| 978.7 | | | | | | | | | | coding | |
| ENST00000379 | 9 | 32455705 | 32526324 | − | DDX58-202 | DDX58 | 4353 | 1.77622664 | 2.12E−07 | protein- | hg38 |
| 883.2 | | | | | | | | | | coding | |
| ENST00000324 | 16 | 28532708 | 28539174 | − | NUPR1-201 | NUPR1 | 5491 | 1.755858563 | 4.32E−09 | protein- | hg38 |
| 873.7 | | | | | | | | | | coding | |
| ENST00000613 | 4 | 23795339 | 23881292 | − | PPARGC1A- | PPARGC1A | 3210 | 1.748036663 | 2.79E−05 | protein- | hg38 |
| 098.4 | | | | | 217 | | | | | coding | |
| ENST00000512 | 5 | 69365357 | 69369477 | − | TAF9-208 | TAF9 | 582 | 1.742712635 | 0.047163 | protein- | hg38 |
| 152.5 | | | | | | | | | | coding | |
| ENST00000252 | 19 | 1397026 | 1401553 | − | GAMT-201 | GAMT | 1121 | 1.732524279 | 0.00017 | protein- | hg38 |
| 288.7 | | | | | | | | | | coding | |
| ENST00000640 | 1 | 99970013 | 100023453 | + | SLC35A3- | SLC35A3 | 1989 | 1.714093381 | 0.002019 | protein- | hg38 |
| 715.1 | | | | | 222 | | | | | coding | |
| ENST00000392 | 12 | 112978402 | 113011718 | − | OAS2-202 | OAS2 | 4734 | 1.705468162 | 6.08E−06 | protein- | hg38 |
| 583.6 | | | | | | | | | | coding | |
| ENST00000219 | 16 | 57256097 | 57284687 | − | PLLP-201 | PLLP | 1512 | 1.698795874 | 4.23E−06 | protein- | hg38 |
| 207.9 | | | | | | | | | | coding | |
| ENST00000443 | 1 | 193018622 | 193029309 | − | UCHL5- | UCHL5 | 785 | 1.692233256 | 0.03335 | protein- | hg38 |
| 327.5 | | | | | 211 | | | | | coding | |
| ENST00000415 | 13 | 21378701 | 21459369 | − | ZDHHC20- | ZDHHC20 | 1296 | 1.669976949 | 0.026944 | protein- | hg38 |
| 724.2 | | | | | 204 | | | | | coding | |
| ENST00000228 | 12 | 112938352 | 112973249 | + | OAS3-201 | OAS3 | 6719 | 1.660466063 | 5.1E−09 | protein- | hg38 |
| 928.11 | | | | | | | | | | coding | |
| ENST00000620 | 12 | 121020292 | 121039242 | − | OASL-204 | OASL | 1695 | 1.658981771 | 0.024008 | protein- | hg38 |
| 239.4 | | | | | | | | | | coding | |
| ENST00000438 | 6 | 125919224 | 125931111 | + | NCOA7- | NCOA7 | 3133 | 1.658474127 | 4.92E−07 | protein- | hg38 |
| 495.6 | | | | | 208 | | | | | coding | |
| ENST00000425 | 7 | 44219213 | 44225913 | − | CAMK2B- | CAMK2B | 867 | 1.657370738 | 0.000399 | protein- | hg38 |
| 809.5 | | | | | 213 | | | | | coding | |
| ENST00000370 | 1 | 97077743 | 97921023 | − | DPYD-202 | DPYD | 4412 | 1.65599767 | 5.94E−08 | protein- | hg38 |
| 192.7 | | | | | | | | | | coding | |
| ENST00000371 | 10 | 88822132 | 88851818 | − | ANKRD22- | ANKRD22 | 1596 | 1.652980745 | 0.000514 | protein- | hg38 |
| 930.4 | | | | | 201 | | | | | coding | |
| ENST00000494 | 17 | 19737984 | 19748393 | − | ALDH3A1- | ALDH3A1 | 1572 | 1.647970653 | 0.032054 | protein- | hg38 |
| 157.6 | | | | | 212 | | | | | coding | |
| ENST00000418 | 1 | 6440378 | 6445757 | + | ESPN-203 | ESPN | 641 | 1.647628063 | 0.005929 | protein- | hg38 |
| 286.1 | | | | | | | | | | coding | |
| ENST00000648 | 4 | 147480932 | 147544954 | + | EDNRA- | EDNRA | 4135 | 1.638680328 | 2.14E−07 | protein- | hg38 |
| 866.1 | | | | | 208 | | | | | coding | |
| ENST00000344 | 1 | 154405223 | 154466877 | + | IL6R-201 | IL6R | 3217 | 1.633523891 | 0.014262 | protein- | hg38 |
| 086.8 | | | | | | | | | | coding | |
| ENST00000301 | 8 | 142680456 | 142682724 | + | PSCA-201 | PSCA | 1020 | 1.629896176 | 0.000235 | protein- | hg38 |
| 258.4 | | | | | | | | | | coding | |
| ENST00000340 | 7 | 73830863 | 73832693 | − | CLDN4- | CLDN4 | 1831 | 1.629281425 | 1.76E−07 | protein- | hg38 |
| 958.3 | | | | | 201 | | | | | coding | |
| ENST00000261 | 16 | 88643283 | 88651152 | − | CYBA-201 | CYBA | 797 | 1.618187233 | 8.71E−07 | protein- | hg38 |
| 623.7 | | | | | | | | | | coding | |
| ENST00000523 | 1 | 230839621 | 230856036 | − | C1orf198- | C1orf198 | 1041 | 1.617321869 | 0.017922 | protein- | hg38 |
| 410.1 | | | | | 207 | | | | | coding | |
| ENST00000443 | 1 | 112917516 | 112935988 | − | SLC16A1- | SLC16A1 | 1099 | 1.609534212 | 0.000596 | protein- | hg38 |
| 580.5 | | | | | 203 | | | | | coding | |
| ENST00000360 | 10 | 78033863 | 78040697 | + | RPS24-201 | RPS24 | 537 | 1.601511841 | 2.8E−05 | protein- | hg38 |
| 830.9 | | | | | | | | | | coding | |
| ENST00000374 | X | 64185117 | 64205708 | − | AMER1- | AMER1 | 8407 | 1.593936624 | 0.017395 | protein- | hg38 |
| 869.8 | | | | | 202 | | | | | coding | |
| ENST00000614 | 7 | 114922417 | 115015935 | + | MDFIC-208 | MDFIC | 1068 | 1.59251307 | 0.021045 | protein- | hg38 |
| 186.5 | | | | | | | | | | coding | |
| ENST00000379 | 7 | 93099516 | 93118023 | − | SAMD9- | SAMD9 | 6852 | 1.589856365 | 1.21E−05 | protein- | hg38 |
| 958.2 | | | | | 201 | | | | | coding | |
| ENST00000594 | 19 | 39445593 | 39457740 | + | SUPT5H- | SUPT5H | 364 | 1.588717114 | 0.022985 | protein- | hg38 |
| 729.5 | | | | | 206 | | | | | coding | |
| ENST00000310 | 1 | 115642629 | 115691854 | + | VANGL1- | VANGL1 | 2265 | 1.572827396 | 0.031919 | protein- | hg38 |
| 260.7 | | | | | 201 | | | | | coding | |
| ENST00000469 | 1 | 224227369 | 224330138 | − | NVL-211 | NVL | 2566 | 1.572530083 | 0.018755 | protein- | hg38 |
| 075.5 | | | | | | | | | | coding | |
| ENST00000512 | 4 | 182144690 | 182346929 | + | TENM3- | TENM3 | 651 | 1.572391594 | 0.009256 | protein- | hg38 |
| 480.5 | | | | | 205 | | | | | coding | |
| ENST00000326 | 5 | 149141483 | 149260542 | + | ABLIM3- | ABLIM3 | 4164 | 1.563527883 | 9E−08 | protein- | hg38 |
| 685.11 | | | | | 202 | | | | | coding | |
| ENST00000464 | 6 | 41067146 | 41072534 | − | OARD1- | OARDI | 717 | 1.547002918 | 0.005024 | protein- | hg38 |
| 633.5 | | | | | 204 | | | | | coding | |
| ENST00000271 | 1 | 151511397 | 151538692 | + | CGN-201 | CGN | 5091 | 1.542030925 | 1.09E−07 | protein- | hg38 |
| 636.11 | | | | | | | | | | coding | |
| ENST00000559 | 15 | 88638953 | 88655511 | + | ISG20-208 | ISG20 | 614 | 1.526307851 | 0.00047 | protein- | hg38 |
| 876.1 | | | | | | | | | | coding | |
| ENST00000504 | 5 | 149141821 | 149260439 | + | ABLIM3- | ABLIM3 | 2774 | 1.518185294 | 7.48E−06 | protein- | hg38 |
| 238.5 | | | | | 205 | | | | | coding | |
| ENST00000374 | 6 | 32854161 | 32859585 | + | PSMB9-207 | PSMB9 | 782 | 1.51627083 | 0.001641 | protein- | hg38 |
| 859.2 | | | | | | | | | | coding | |
| ENST00000273 | 3 | 99638596 | 99796733 | + | COL8A1- | COL8A1 | 3029 | 1.515424377 | 0.001368 | protein- | hg38 |
| 342.8 | | | | | 202 | | | | | coding | |
| ENST00000415 | 6 | 41934956 | 42048894 | − | CCND3- | CCND3 | 1843 | 1.515421084 | 0.000802 | protein- | hg38 |
| 497.6 | | | | | 205 | | | | | coding | |
| ENST00000498 | 9 | 21968105 | 21995301 | − | CDKN2A- | CDKN2A | 926 | 1.511139218 | 0.003044 | protein- | hg38 |
| 628.6 | | | | | 209 | | | | | coding | |
| ENST00000648 | 8 | 47960898 | 47977016 | + | MCM4-217 | MCM4 | 2598 | 1.502554904 | 0.043114 | protein- | hg38 |
| 407.1 | | | | | | | | | | coding | |
| ENST00000553 | 12 | 112916617 | 112919210 | + | OAS1-210 | OAS1 | 890 | 1.499731073 | 0.002831 | protein- | hg38 |
| 152.1 | | | | | | | | | | coding | |
| ENST00000591 | 12 | 53542887 | 53626410 | − | ATF7-212 | ATF7 | 860 | 1.483036353 | 0.04324 | protein- | hg38 |
| 397.1 | | | | | | | | | | coding | |
| ENST00000393 | 14 | 94612384 | 94624055 | + | SERPINA3- | SERPINA3 | 1581 | 1.480391181 | 0.023728 | protein- | hg38 |
| 080.8 | | | | | 202 | | | | | coding | |
| ENST00000372 | 10 | 86958656 | 86963260 | + | SNCG-202 | SNCG | 701 | 1.475187319 | 2.67E−05 | protein- | hg38 |
| 017.3 | | | | | | | | | | coding | |
| ENST00000434 | 19 | 281040 | 291504 | − | PLPP2-203 | PLPP2 | 1383 | 1.467331132 | 9.09E−06 | protein- | hg38 |
| 325.6 | | | | | | | | | | coding | |
| ENST00000269 | 17 | 82321024 | 82333998 | − | SECTMI- | SECTM1 | 2235 | 1.465509382 | 1.52E−07 | protein- | hg38 |
| 389.7 | | | | | 201 | | | | | coding | |
| ENST00000252 | 19 | 18386158 | 18389176 | + | GDF15-201 | GDF15 | 1200 | 1.464416629 | 1.55E−07 | protein- | hg38 |
| 809.3 | | | | | | | | | | coding | |
| ENST00000358 | 22 | 24181259 | 24189110 | + | SUSD2-201 | SUSD2 | 3404 | 1.463546418 | 7.01E−07 | protein- | hg38 |
| 321.3 | | | | | | | | | | coding | |
| ENST00000276 | 9 | 19115770 | 19127576 | − | PLIN2-201 | PLIN2 | 1972 | 1.45802159 | 2.93E−07 | protein- | hg38 |
| 914.6 | | | | | | | | | | coding | |
| ENST00000437 | 5 | 96741342 | 96774683 | + | CAST-209 | CAST | 3377 | 1.448825548 | 2.3E−05 | protein- | hg38 |
| 034.6 | | | | | | | | | | coding | |
| ENST00000355 | 6 | 133241357 | 133532119 | + | EYA4-201 | EYA4 | 5692 | 1.441577404 | 0.034804 | protein- | hg38 |
| 167.7 | | | | | | | | | | coding | |
| ENST00000370 | 1 | 75202131 | 75611116 | − | SLC44A5- | SLC44A5 | 3896 | 1.438088146 | 1.39E−07 | protein- | hg38 |
| 859.7 | | | | | 202 | | | | | coding | |
| ENST00000202 | 12 | 112906777 | 112919903 | + | OAS1-201 | OAS1 | 1816 | 1.42445533 | 0.001181 | protein- | hg38 |
| 917.9 | | | | | | | | | | coding | |
| ENST00000485 | 3 | 111071743 | 111135954 | + | NECTIN3- | NECTIN3 | 3664 | 1.413966917 | 5.53E−05 | protein- | hg38 |
| 303.5 | | | | | 206 | | | | | coding | |
| ENST00000606 | 1 | 150487420 | 150507284 | + | TARS2-214 | TARS2 | 2162 | 1.407383787 | 0.043118 | protein- | hg38 |
| 933.5 | | | | | | | | | | coding | |
| ENST00000323 | 2 | 201260500 | 201287709 | + | CASP8-203 | CASP8 | 2650 | 1.406496761 | 0.002499 | protein- | hg38 |
| 492.11 | | | | | | | | | | coding | |
| ENST00000423 | 19 | 45407334 | 45478828 | − | ERCC1-204 | ERCC1 | 3119 | 1.389032771 | 0.018079 | protein- | hg38 |
| 698.6 | | | | | | | | | | coding | |
| ENST00000287 | 11 | 57551662 | 57567807 | − | UBE2L6- | UBE2L6 | 1354 | 1.38472936 | 5.15E−05 | protein- | hg38 |
| 156.8 | | | | | 201 | | | | | coding | |
| ENST00000448 | 3 | 146515955 | 146544620 | − | PLSCR1- | PLSCR1 | 996 | 1.380290587 | 1.39E−05 | protein- | hg38 |
| 787.6 | | | | | 202 | | | | | coding | |
| ENST00000511 | 5 | 80628124 | 80654552 | − | DHFR-205 | DHFR | 1474 | 1.363387586 | 0.038267 | protein- | hg38 |
| 032.5 | | | | | | | | | | coding | |
| ENST00000342 | 11 | 64823387 | 64844569 | − | CDC42BPG- | CDC42BPG | 5742 | 1.361107963 | 9.98E−08 | protein- | hg38 |
| 711.5 | | | | | 201 | | | | | coding | |
| ENST00000438 | 17 | 43006740 | 43014456 | + | IFI35-204 | IFI35 | 1232 | 1.354485215 | 0.017385 | protein- | hg38 |
| 323.2 | | | | | | | | | | coding | |
| ENST00000370 | 1 | 86424086 | 86456558 | + | CLCA2-201 | CLCA2 | 4025 | 1.349992624 | 8.14E−07 | protein- | hg38 |
| 565.4 | | | | | | | | | | coding | |
| ENST00000471 | 7 | 139060338 | 139109719 | − | ZC3HAV1- | ZC3HAV1 | 3182 | 1.343526307 | 1.14E−05 | protein- | hg38 |
| 652.1 | | | | | 204 | | | | | coding | |
| ENST00000222 | 7 | 2519842 | 2528429 | + | LFNG-201 | LFNG | 2377 | 1.336747581 | 0.000258 | protein- | hg38 |
| 725.9 | | | | | | | | | | coding | |
| ENST00000591 | 19 | 45409619 | 45423501 | − | ERCC1-212 | ERCC1 | 836 | 1.33584231 | 0.024657 | protein- | hg38 |
| 636.5 | | | | | | | | | | coding | |
| ENST00000551 | 12 | 98593650 | 98601707 | + | SLC25A3- | SLC25A3 | 1359 | 1.335564474 | 0.024824 | protein- | hg38 |
| 917.5 | | | | | 216 | | | | | coding | |
| ENST00000496 | 11 | 3808594 | 3826330 | + | PGAP2-227 | PGAP2 | 1530 | 1.332402007 | 0.02567 | protein- | hg38 |
| 834.6 | | | | | | | | | | coding | |
| ENST00000339 | 2 | 187478585 | 187554438 | − | TFPI-202 | TFPI | 1088 | 1.33234038 | 0.010318 | protein- | hg38 |
| 091.8 | | | | | | | | | | coding | |
| ENST00000360 | 3 | 146069444 | 146161167 | − | PLOD2-202 | PLOD2 | 3665 | 1.326148178 | 5.56E−06 | protein- | hg38 |
| 060.7 | | | | | | | | | | coding | |
| ENST00000562 | 15 | 72209751 | 72222531 | − | PKM-207 | PKM | 582 | 1.325764124 | 0.020252 | protein- | hg38 |
| 997.5 | | | | | | | | | | coding | |
| ENST00000438 | 1 | 78649832 | 78659428 | + | IFI44-202 | IFI44 | 686 | 1.324565046 | 0.010525 | protein- | hg38 |
| 486.1 | | | | | | | | | | coding | |
| ENST00000647 | 12 | 56714612 | 56741535 | − | AC117378.1- | AC117378.1 | 588 | 1.321838867 | 0.047677 | protein- | hg38 |
| 707.1 | | | | | 201 | | | | | coding | |
| ENST00000579 | 9 | 21967753 | 21994624 | − | CDKN2A- | CDKN2A | 1283 | 1.321645272 | 0.000834 | protein- | hg38 |
| 755.1 | | | | | 214 | | | | | coding | |
| ENST00000615 | 1 | 239632206 | 239909415 | + | CHRM3- | CHRM3 | 2294 | 1.320580373 | 8.26E−05 | protein- | hg38 |
| 928.4 | | | | | 207 | | | | | coding | |
| ENST00000373 | 1 | 29236516 | 29326800 | + | PTPRU-202 | PTPRU | 5579 | 1.319349123 | 1.15E−06 | protein- | hg38 |
| 779.7 | | | | | | | | | | coding | |
| ENST00000420 | 1 | 75724780 | 75762809 | + | ACADM- | ACADM | 1332 | 1.317979264 | 0.000638 | protein- | hg38 |
| 607.6 | | | | | 203 | | | | | coding | |
| ENST00000371 | 10 | 88879734 | 88923487 | + | STAMBPL1- | STAMBPL1 | 2532 | 1.313560467 | 2.74E−06 | protein- | hg38 |
| 926.7 | | | | | 203 | | | | | coding | |
| ENST00000553 | 16 | 14750813 | 14765413 | + | NPIPA2- | NPIPA2 | 1053 | 1.310591989 | 0.003994 | protein- | hg38 |
| 201.1 | | | | | 203 | | | | | coding | |
| ENST00000378 | X | 30653359 | 30730608 | + | GK-203 | GK | 3707 | 1.309416479 | 2.01E−05 | protein- | hg38 |
| 943.7 | | | | | | | | | | coding | |
| ENST00000591 | 17 | 44345302 | 44350283 | + | GRN-218 | GRN | 585 | 1.306794267 | 0.041062 | protein- | hg38 |
| 740.5 | | | | | | | | | | coding | |
| ENST00000333 | 22 | 38982409 | 38992784 | + | APOBEC3B- | APOBEC3B | 1533 | 1.3040769 | 9.46E−05 | protein- | hg38 |
| 467.3 | | | | | 201 | | | | | coding | |
| ENST00000262 | X | 85277396 | 85379743 | − | POF1B-201 | POF1B | 3941 | 1.302427429 | 1.7E−06 | protein- | hg38 |
| 753.8 | | | | | | | | | | coding | |
| ENST00000646 | 1 | 99708632 | 99766630 | − | FRRS1-205 | FRRS1 | 2304 | 1.300571247 | 3.16E−06 | protein- | hg38 |
| 001.1 | | | | | | | | | | coding | |
| ENST00000507 | 6 | 99464636 | 99503773 | − | USP45-210 | USP45 | 715 | 1.299531643 | 0.014179 | protein- | hg38 |
| 717.5 | | | | | | | | | | coding | |
| ENST00000360 | 20 | 1309975 | 1329239 | − | SDCBP2- | SDCBP2 | 1519 | 1.297179778 | 0.000382 | protein- | hg38 |
| 779.3 | | | | | 202 | | | | | coding | |
| ENST00000371 | 10 | 89205629 | 89207314 | − | CH25H-201 | CH25H | 1686 | 1.296271652 | 0.001245 | protein- | hg38 |
| 852.3 | | | | | | | | | | coding | |
| ENST00000343 | 16 | 23302270 | 23381299 | + | SCNNIB- | SCNN1B | 2597 | 1.290633396 | 0.003293 | protein- | hg38 |
| 070.6 | | | | | 202 | | | | | coding | |
| ENST00000245 | 19 | 6677704 | 6720682 | − | C3-201 | C3 | 5263 | 1.282167524 | 1.71E−08 | protein- | hg38 |
| 907.10 | | | | | | | | | | coding | |
| ENST00000263 | 11 | 102317495 | 102337734 | + | BIRC3-201 | BIRC3 | 5197 | 1.279393292 | 3.07E−06 | protein- | hg38 |
| 464.7 | | | | | | | | | | coding | |
| ENST00000335 | 11 | 65787022 | 65797219 | + | OVOL1- | OVOL1 | 3034 | 1.279364688 | 1.83E−06 | protein- | hg38 |
| 987.7 | | | | | 201 | | | | | coding | |
| ENST00000412 | 6 | 31353872 | 31357187 | − | HLA-B-249 | HLA-B | 1547 | 1.276259013 | 2.28E−08 | protein- | hg38 |
| 585.6 | | | | | | | | | | coding | |
| ENST00000338 | 2 | 237487251 | 237553994 | + | MLPH-202 | MLPH | 2332 | 1.276096482 | 0.025254 | protein- | hg38 |
| 530.8 | | | | | | | | | | coding | |
| ENST00000276 | 9 | 22002903 | 22009363 | − | CDKN2B- | CDKN2B | 3911 | 1.272444725 | 2.82E−08 | protein- | hg38 |
| 925.6 | | | | | 201 | | | | | coding | |
| ENST00000444 | X | 153786801 | 153794359 | − | IDH3G-206 | IDH3G | 888 | 1.271104595 | 0.028463 | protein- | hg38 |
| 450.5 | | | | | | | | | | coding | |
| ENST00000555 | 14 | 75278977 | 75280789 | + | FOS-205 | FOS | 629 | 1.26536441 | 0.015278 | protein- | hg38 |
| 242.1 | | | | | | | | | | coding | |
| ENST00000368 | 1 | 156699606 | 156705601 | − | CRABP2- | CRABP2 | 992 | 1.265023982 | 0.000735 | protein- | hg38 |
| 222.7 | | | | | 203 | | | | | coding | |
| ENST00000312 | 11 | 66011841 | 66013505 | − | CST6-201 | CST6 | 759 | 1.263773971 | 0.000842 | protein- | hg38 |
| 134.2 | | | | | | | | | | coding | |
| ENST00000325 | 4 | 41935152 | 41960041 | − | TMEM33- | TMEM33 | 6221 | 1.259795926 | 0.025456 | protein- | hg38 |
| 094.9 | | | | | 202 | | | | | coding | |
| ENST00000265 | 9 | 119166630 | 119369467 | − | BRINP1- | BRINP1 | 3202 | 1.258704104 | 9.42E−07 | protein- | hg38 |
| 922.7 | | | | | 201 | | | | | coding | |
| ENST00000301 | 19 | 8364151 | 8374373 | + | ANGPTL4- | ANGPTL4 | 1879 | 1.255791714 | 0.010142 | protein- | hg38 |
| 455.6 | | | | | 201 | | | | | coding | |
| ENST00000452 | 12 | 112906850 | 112918462 | + | OAS1-203 | OAS1 | 1990 | 1.248296033 | 0.001156 | protein- | hg38 |
| 357.6 | | | | | | | | | | coding | |
| ENST00000237 | 5 | 95813849 | 95823005 | − | GLRX-201 | GLRX | 1211 | 1.245230124 | 1.45E−05 | protein- | hg38 |
| 858.10 | | | | | | | | | | coding | |
| ENST00000262 | 22 | 45502883 | 45563362 | + | FBLN1-201 | FBLN1 | 2251 | 1.244787621 | 6.46E−06 | protein- | hg38 |
| 722.11 | | | | | | | | | | coding | |
| ENST00000560 | 15 | 84669544 | 84716111 | − | SEC11A- | SEC11A | 1089 | 1.240747324 | 0.000145 | protein- | hg38 |
| 266.5 | | | | | 209 | | | | | coding | |
| ENST00000392 | 2 | 190975537 | 191014168 | − | STAT1-202 | STAT1 | 2716 | 1.238694659 | 0.000586 | protein- | hg38 |
| 322.7 | | | | | | | | | | coding | |
| ENST00000563 | 16 | 30064274 | 30070414 | + | ALDOA- | ALDOA | 1550 | 1.238121527 | 9.35E−05 | protein- | hg38 |
| 060.6 | | | | | 206 | | | | | coding | |
| ENST00000261 | 3 | 99638475 | 99799226 | + | COL8A1- | COL8A1 | 5705 | 1.23513888 | 5.39E−05 | protein- | hg38 |
| 037.7 | | | | | 201 | | | | | coding | |
| ENST00000380 | 9 | 22005987 | 22009272 | − | CDKN2B- | CDKN2B | 859 | 1.230678296 | 0.000241 | protein- | hg38 |
| 142.4 | | | | | 202 | | | | | coding | |
| ENST00000327 | 22 | 45502891 | 45601135 | + | FBLN1-202 | FBLN1 | 2896 | 1.229023581 | 1.72E−06 | protein- | hg38 |
| 858.10 | | | | | | | | | | coding | |
| ENST00000453 | 2 | 187496884 | 187554492 | − | TFPI-210 | TFPI | 733 | 1.226250225 | 0.007553 | protein- | hg38 |
| 013.5 | | | | | | | | | | coding | |
| ENST00000361 | 14 | 69879416 | 70030727 | + | SMOC1- | SMOC1 | 2040 | 1.224751421 | 1.67E−06 | protein- | hg38 |
| 956.7 | | | | | 201 | | | | | coding | |
| ENST00000381 | 11 | 1838989 | 1841678 | + | TNNI2-204 | TNNI2 | 743 | 1.219014418 | 0.004078 | protein- | hg38 |
| 911.5 | | | | | | | | | | coding | |
| ENST00000261 | 1 | 114717295 | 114757974 | − | CSDE1-201 | CSDE1 | 3228 | 1.212502489 | 0.000214 | protein- | hg38 |
| 443.9 | | | | | | | | | | coding | |
| ENST00000358 | 11 | 47468284 | 47489014 | − | CELF1-202 | CELF1 | 2108 | 1.20923745 | 0.043583 | protein- | hg38 |
| 597.7 | | | | | | | | | | coding | |
| ENST00000381 | 14 | 69879426 | 70032366 | + | SMOC1- | SMOC1 | 3666 | 1.204365528 | 3.49E−06 | protein- | hg38 |
| 280.4 | | | | | 202 | | | | | coding | |
| ENST00000252 | 2 | 1631887 | 1744506 | − | PXDN-201 | PXDN | 6808 | 1.202957804 | 1.01E−08 | protein- | hg38 |
| 804.8 | | | | | | | | | | coding | |
| ENST00000359 | 1 | 110004131 | 110022389 | + | AHCYL1- | AHCYL1 | 2503 | 1.200589992 | 0.00205 | protein- | hg38 |
| 172.3 | | | | | 201 | | | | | coding | |
| ENST00000638 | 1 | 99970024 | 100015697 | + | SLC35A3- | SLC35A3 | 1286 | 1.198206287 | 0.008926 | protein- | hg38 |
| 988.1 | | | | | 213 | | | | | coding | |
| ENST00000404 | 7 | 12687635 | 12688914 | + | ARL4A- | ARL4A | 840 | 1.186301266 | 0.001906 | protein- | hg38 |
| 894.1 | | | | | 205 | | | | | coding | |
| ENST00000268 | 17 | 73232637 | 73248874 | + | C17orf80- | C17orf80 | 3449 | 1.184459936 | 0.04443 | protein- | hg38 |
| 942.12 | | | | | 202 | | | | | coding | |
| ENST00000308 | 1 | 204198160 | 204214092 | − | GOLT1A- | GOLT1A | 883 | 1.179869656 | 0.009353 | protein- | hg38 |
| 302.3 | | | | | 201 | | | | | coding | |
| ENST00000370 | 1 | 88935773 | 88992776 | − | KYAT3- | KYAT3 | 1868 | 1.178914559 | 1.72E−05 | protein- | hg38 |
| 491.7 | | | | | 203 | | | | | coding | |
| ENST00000267 | 14 | 24239643 | 24242674 | − | TINF2-201 | TINF2 | 1852 | 1.174885412 | 0.046248 | protein- | hg38 |
| 415.11 | | | | | | | | | | coding | |
| ENST00000378 | X | 30653478 | 30729170 | + | GK-204 | GK | 2063 | 1.161979694 | 0.000379 | protein- | hg38 |
| 945.7 | | | | | | | | | | coding | |
| ENST00000306 | 4 | 76033682 | 76036197 | − | CXCL11- | CXCL11 | 1606 | 1.159735131 | 0.001149 | protein- | hg38 |
| 621.7 | | | | | 201 | | | | | coding | |
| ENST00000340 | 19 | 43648580 | 43670350 | − | PLAUR- | PLAUR | 1548 | 1.158213455 | 5.48E−07 | protein- | hg38 |
| 093.7 | | | | | 203 | | | | | coding | |
| ENST00000358 | X | 81113701 | 81201942 | − | HMGN5- | HMGN5 | 2126 | 1.150438455 | 0.009353 | protein- | hg38 |
| 130.6 | | | | | 201 | | | | | coding | |
| ENST00000607 | 1 | 152804835 | 152805478 | − | LCE1C-202 | LCE1C | 644 | 1.148019512 | 0.032191 | protein- | hg38 |
| 093.1 | | | | | | | | | | coding | |
| ENST00000471 | 3 | 122528005 | 122564242 | − | PARP9-204 | PARP9 | 3040 | 1.147220938 | 0.002751 | protein- | hg38 |
| 785.5 | | | | | | | | | | coding | |
| ENST00000345 | 8 | 66793614 | 66862022 | + | SGK3-201 | SGK3 | 4055 | 1.147047433 | 0.046262 | protein- | hg38 |
| 714.8 | | | | | | | | | | coding | |
| ENST00000422 | 17 | 40019503 | 40023160 | − | MED24- | MED24 | 905 | 1.143191754 | 0.003406 | protein- | hg38 |
| 942.6 | | | | | 205 | | | | | coding | |
| ENST00000370 | 1 | 167541013 | 167553767 | − | CREG1-201 | CREG1 | 1974 | 1.141436248 | 1.58E−05 | protein- | hg38 |
| 509.4 | | | | | | | | | | coding | |
| ENST00000646 | 4 | 153684070 | 153703646 | + | TLR2-209 | TLR2 | 1177 | 1.136662666 | 0.01496 | protein- | hg38 |
| 900.1 | | | | | | | | | | coding | |
| ENST00000244 | 6 | 56056590 | 56247746 | − | COL21A1- | COL21A1 | 4339 | 1.124118493 | 0.012067 | protein- | hg38 |
| 728.9 | | | | | 201 | | | | | coding | |
| ENST00000437 | 5 | 132485667 | 132490777 | − | IRF1-203 | IRF1 | 832 | 1.122842161 | 0.000482 | protein- | hg38 |
| 654.5 | | | | | | | | | | coding | |
| ENST00000591 | 17 | 78971238 | 78979918 | − | LGALS3BP- | LGALS3BP | 1961 | 1.118288168 | 0.03995 | protein- | hg38 |
| 778.5 | | | | | 218 | | | | | coding | |
| ENST00000305 | 3 | 149369022 | 149377865 | − | TM4SF1- | TM4SF1 | 1771 | 1.11727016 | 1.83E−05 | protein- | hg38 |
| 366.7 | | | | | 201 | | | | | coding | |
| ENST00000251 | 17 | 42101404 | 42112733 | − | DHX58-201 | DHX58 | 2617 | 1.116404153 | 0.009189 | protein- | hg38 |
| 642.7 | | | | | | | | | | coding | |
| ENST00000371 | 1 | 58575423 | 58577773 | − | TACSTD2- | TACSTD2 | 2351 | 1.115708016 | 7.42E−07 | protein- | hg38 |
| 225.3 | | | | | 201 | | | | | coding | |
| ENST00000288 | 14 | 24290598 | 24299833 | − | DHRS1-201 | DHRS1 | 1480 | 1.107297824 | 1.48E−05 | protein- | hg38 |
| 111.11 | | | | | | | | | | coding | |
| ENST00000306 | 15 | 88638743 | 88656483 | + | ISG20-201 | ISG20 | 1856 | 1.105778034 | 7.83E−05 | protein- | hg38 |
| 072.9 | | | | | | | | | | coding | |
| ENST00000260 | 15 | 56428731 | 56465137 | − | MNS1-201 | MNS1 | 2023 | 1.105147615 | 1.45E−05 | protein- | hg38 |
| 453.3 | | | | | | | | | | coding | |
| ENST00000530 | 9 | 21968001 | 21994411 | − | CDKN2A- | CDKN2A | 748 | 1.104877994 | 8.31E−05 | protein- | hg38 |
| 628.2 | | | | | 210 | | | | | coding | |
| ENST00000306 | 1 | 98661723 | 98760500 | + | SNX7-201 | SNX7 | 1734 | 1.103171223 | 3.22E−06 | protein- | hg38 |
| 121.7 | | | | | | | | | | coding | |
| ENST00000555 | 12 | 57230354 | 57231913 | + | SHMT2- | SHMT2 | 600 | 1.099055502 | 0.009523 | protein- | hg38 |
| 773.5 | | | | | 221 | | | | | coding | |
| ENST00000525 | 11 | 44933036 | 44950874 | − | TP53111- | TP53I11 | 2647 | 1.097814404 | 0.048493 | protein- | hg38 |
| 680.5 | | | | | 208 | | | | | coding | |
| ENST00000637 | 15 | 51056604 | 51094705 | − | TNFAIP8L3- | TNFAIP8L3 | 2002 | 1.096883624 | 0.001904 | protein- | hg38 |
| 513.1 | | | | | 202 | | | | | coding | |
| ENST00000377 | 1 | 7919847 | 7940866 | − | TNFRSF9- | TNFRSF9 | 1923 | 1.092434453 | 0.000272 | protein- | hg38 |
| 507.7 | | | | | 201 | | | | | coding | |
| ENST00000421 | X | 107153292 | 107206433 | | NUP62CL- | NUP62CL | 618 | 1.091444621 | 0.017065 | protein- | hg38 |
| 752.1 | | | | | 202 | | | | | coding | |
| ENST00000398 | 11 | 67583595 | 67586656 | + | GSTP1-202 | GSTP1 | 961 | 1.088616726 | 0.000386 | protein- | hg38 |
| 606.8 | | | | | | | | | | coding | |
| ENST00000565 | X | 136873978 | 136880764 | − | RBMX-209 | RBMX | 1292 | 1.086826055 | 0.001733 | protein- | hg38 |
| 438.1 | | | | | | | | | | coding | |
| ENST00000474 | 3 | 122680618 | 122730840 | + | PARP14- | PARP14 | 7915 | 1.086260872 | 1.01E−06 | protein- | hg38 |
| 629.6 | | | | | 202 | | | | | coding | |
| ENST00000376 | 9 | 82979585 | 83063128 | − | RASEF-202 | RASEF | 5576 | 1.084944545 | 6.62E−07 | protein- | hg38 |
| 447.3 | | | | | | | | | | coding | |
| ENST00000433 | 1 | 111619777 | 111704405 | + | RAP1A-203 | RAP1A | 666 | 1.080799519 | 0.001482 | protein- | hg38 |
| 097.5 | | | | | | | | | | coding | |
| ENST00000592 | 17 | 78378670 | 78403679 | − | PGS1-215 | PGS1 | 988 | 1.079873937 | 0.048509 | protein- | hg38 |
| 043.5 | | | | | | | | | | coding | |
| ENST00000357 | 1 | 112674745 | 112700710 | − | MOV10- | MOV10 | 3383 | 1.079809777 | 3.51E−05 | protein- | hg38 |
| 443.2 | | | | | 201 | | | | | coding | |
| ENST00000379 | 16 | 69709401 | 69726668 | − | NQO1-203 | NQO1 | 2527 | 1.079527383 | 0.000151 | protein- | hg38 |
| 047.7 | | | | | | | | | | coding | |
| ENST00000267 | 12 | 121777754 | 121794262 | − | RHOF-201 | RHOF | 3009 | 1.076945194 | 9E−07 | protein- | hg38 |
| 205.6 | | | | | | | | | | coding | |
| ENST00000405 | 5 | 132483086 | 132490262 | − | IRF1-202 | IRF1 | 2061 | 1.074349622 | 7.61E−06 | protein- | hg38 |
| 885.6 | | | | | | | | | | coding | |
| ENST00000310 | 4 | 114598455 | 114678224 | + | UGT8-201 | UGT8 | 4084 | 1.072205509 | 0.000112 | protein- | hg38 |
| 836.10 | | | | | | | | | | coding | |
| ENST00000370 | 1 | 84498329 | 84506565 | − | GNG5-201 | GNG5 | 920 | 1.069911739 | 0.004097 | protein- | hg38 |
| 641.3 | | | | | | | | | | coding | |
| ENST00000392 | 6 | 122610232 | 122726372 | + | PKIB-206 | PKIB | 1811 | 1.069629832 | 0.02107 | protein- | hg38 |
| 490.5 | | | | | | | | | | coding | |
| ENST00000318 | 11 | 26994184 | 26996121 | + | FIBIN-201 | FIBIN | 1938 | 1.066399894 | 0.000432 | protein- | hg38 |
| 627.3 | | | | | | | | | | coding | |
| ENST00000371 | 1 | 56645322 | 56715335 | + | PRKAA2- | PRKAA2 | 9347 | 1.065173779 | 0.009315 | protein- | hg38 |
| 244.8 | | | | | 201 | | | | | coding | |
| ENST00000352 | 11 | 64318182 | 64321740 | + | PRDX5-203 | PRDX5 | 596 | 1.064284882 | 0.00028 | protein- | hg38 |
| 435.8 | | | | | | | | | | coding | |
| ENST00000255 | 11 | 63998558 | 64166061 | − | MACROD1- | MACROD1 | 1205 | 1.064039026 | 1.99E−05 | protein- | hg38 |
| 681.6 | | | | | 201 | | | | | coding | |
| ENST00000467 | 20 | 63559202 | 63572455 | − | HELZ2-204 | HELZ2 | 8064 | 1.060676814 | 1.44E−06 | protein- | hg38 |
| 148.1 | | | | | | | | | | coding | |
| ENST00000589 | 19 | 5842891 | 5851474 | − | FUT3-207 | FUT3 | 2239 | 1.057696804 | 0.0007 | protein- | hg38 |
| 620.5 | | | | | | | | | | coding | |
| ENST00000369 | 20 | 63974113 | 63979642 | − | SAMD10- | SAMD10 | 2181 | 1.05730068 | 7.39E−05 | protein- | hg38 |
| 886.7 | | | | | 201 | | | | | coding | |
| ENST00000409 | 2 | 197453493 | 197474168 | − | COQ10B- | COQ10B | 879 | 1.055791847 | 0.047926 | protein- | hg38 |
| 398.5 | | | | | 203 | | | | | coding | |
| ENST00000354 | 11 | 494552 | 507221 | − | RNH1-201 | RNH1 | 1894 | 1.055540263 | 0.001294 | protein- | hg38 |
| 420.6 | | | | | | | | | | coding | |
| ENST00000376 | 6 | 29942245 | 29945884 | − | HLA-A-202 | HLA-A | 1854 | 1.054755771 | 1.08E−05 | protein- | hg38 |
| 806.9 | | | | | | | | | | coding | |
| ENST00000206 | 6 | 153010722 | 153131249 | − | RGS17-201 | RGS17 | 1636 | 1.050812352 | 0.001916 | protein- | hg38 |
| 262.1 | | | | | | | | | | coding | |
| ENST00000550 | 12 | 112907052 | 112916816 | + | OAS1-206 | OAS1 | 950 | 1.045057082 | 0.016027 | protein- | hg38 |
| 689.1 | | | | | | | | | | coding | |
| ENST00000607 | 15 | 36895149 | 37095021 | − | MEIS2-227 | MEIS2 | 705 | 1.044547999 | 0.033916 | protein- | hg38 |
| 277.5 | | | | | | | | | | coding | |
| ENST00000271 | 1 | 150549369 | 150560932 | + | ADAMTSL4- | ADAMTSL4 | 4250 | 1.044039475 | 3.6E−05 | protein- | hg38 |
| 643.8 | | | | | 201 | | | | | coding | |
| ENST00000370 | 1 | 77695987 | 77759852 | − | USP33-204 | USP33 | 4327 | 1.041177296 | 0.000181 | protein- | hg38 |
| 794.7 | | | | | | | | | | coding | |
| ENST00000264 | 19 | 10270835 | 10286615 | + | ICAM1-201 | ICAM1 | 3252 | 1.040535278 | 7.64E−05 | protein- | hg38 |
| 832.7 | | | | | | | | | | coding | |
| ENST00000319 | 7 | 29563811 | 29567295 | + | PRR15-201 | PRR15 | 1678 | 1.035061933 | 2.22E−06 | protein- | hg38 |
| 694.2 | | | | | | | | | | coding | |
| ENST00000359 | 5 | 107859035 | 108381410 | − | FBXL17- | FBXL17 | 4510 | 1.032579004 | 0.000155 | protein- | hg38 |
| 660.9 | | | | | 201 | | | | | coding | |
| ENST00000255 | 11 | 63552770 | 63563383 | − | HRASLS2- | HRASLS2 | 742 | 1.032565689 | 0.008265 | protein- | hg38 |
| 695.1 | | | | | 201 | | | | | coding | |
| ENST00000372 | X | 103309346 | 103311046 | − | BEX2-202 | BEX2 | 899 | 1.03209662 | 8.21E−07 | protein- | hg38 |
| 677.7 | | | | | | | | | | coding | |
| ENST00000358 | 16 | 67934502 | 67937087 | − | PSMB10- | PSMB10 | 1218 | 1.02728135 | 0.001303 | protein- | hg38 |
| 514.8 | | | | | 201 | | | | | coding | |
| ENST00000360 | 16 | 29459889 | 29464976 | + | SULTIA4- | SULT1A4 | 1390 | 1.027234521 | 0.03877 | protein- | hg38 |
| 423.11 | | | | | 201 | | | | | coding | |
| ENST00000370 | 1 | 90915298 | 91021473 | − | ZNF644- | ZNF644 | 5702 | 1.024422188 | 0.026572 | protein- | hg38 |
| 440.5 | | | | | 204 | | | | | coding | |
| ENST00000370 | 1 | 100872387 | 100894812 | − | EXTL2-201 | EXTL2 | 2835 | 1.022605206 | 0.000621 | protein- | hg38 |
| 113.7 | | | | | | | | | | coding | |
| ENST00000255 | X | 106726664 | 106796993 | + | RNF128- | RNF128 | 2817 | 1.020463449 | 2.22E−06 | protein- | hg38 |
| 499.2 | | | | | 201 | | | | | coding | |
| ENST00000367 | 1 | 182598623 | 182604408 | − | RGS16-201 | RGS16 | 2427 | 1.018740178 | 2.56E−05 | protein- | hg38 |
| 558.5 | | | | | | | | | | coding | |
| ENST00000352 | 8 | 78516355 | 78603185 | − | PKIA-201 | PKIA | 1736 | 1.017748699 | 0.012685 | protein- | hg38 |
| 966.9 | | | | | | | | | | coding | |
| ENST00000476 | 6 | 167951949 | 167963060 | + | AFDN-212 | AFDN | 867 | 1.016874951 | 0.017701 | protein- | hg38 |
| 946.2 | | | | | | | | | | coding | |
| ENST00000535 | 1 | 86704570 | 86748176 | − | SH3GLB1- | SH3GLB1 | 6227 | 1.014736102 | 0.004191 | protein- | hg38 |
| 010.5 | | | | | 203 | | | | | coding | |
| ENST00000445 | 2 | 119679191 | 119681195 | + | TMEM177- | TMEM177 | 791 | 1.013585088 | 0.045371 | protein- | hg38 |
| 518.1 | | | | | 205 | | | | | coding | |
| ENST00000529 | 8 | 38263130 | 38269140 | − | PLPP5-209 | PLPP5 | 2185 | 1.013525386 | 0.000551 | protein- | hg38 |
| 359.5 | | | | | | | | | | coding | |
| ENST00000368 | 1 | 159009918 | 159055151 | + | IFI16-205 | IFI16 | 2704 | 1.012486902 | 1.69E−05 | protein- | hg38 |
| 132.7 | | | | | | | | | | coding | |
| ENST00000398 | 21 | 43053191 | 43075945 | − | CBS-204 | CBS | 2605 | 1.011467368 | 3.62E−06 | protein- | hg38 |
| 165.7 | | | | | | | | | | coding | |
| ENST00000630 | 1 | 196652045 | 196701566 | + | CFH-206 | CFH | 1658 | 1.011352309 | 0.01858 | protein- | hg38 |
| 130.2 | | | | | | | | | | coding | |
| ENST00000605 | 17 | 35872002 | 35880291 | − | CCL5-203 | CCL5 | 719 | 1.006059812 | 0.007495 | protein- | hg38 |
| 509.1 | | | | | | | | | | coding | |
| ENST00000370 | 1 | 78620403 | 78646145 | − | IFI44L-201 | IFI44L | 5874 | 1.005018412 | 0.000101 | protein- | hg38 |
| 751.9 | | | | | | | | | | coding | |
| ENST00000483 | 1 | 1628489 | 1630589 | + | MIB2-211 | MIB2 | 1058 | 1.001682773 | 0.011238 | protein- | hg38 |
| 015.1 | | | | | | | | | | coding | |

TABLE 3
LncRNA Biomarkers

| | | | | | | | | | p- | | |
|---|---|---|---|---|---|---|---|---|---|---|---|
| enst | chromosome | start.position | end.position | strand | transcript.id | gene | len | log2FoldChange | value | biotype | genome |
| ENST00000514 | 6 | 41937713 | 42048688 | − | CCND3-220 | CCND3 | 476 | 3.788433339 | 0.002192 | lncRNA | hg38 |
| 382.5 | | | | | | | | | | | |
| ENST00000495 | 1 | 78649833 | 78664078 | + | IFI44-209 | IFI44 | 1117 | 2.217078372 | 0.000116 | lncRNA | hg38 |
| 254.5 | | | | | | | | | | | |
| ENST00000545 | 12 | 20855092 | 20861054 | + | SLCO1B3- | SLCO1B3 | 339 | 2.133353419 | 0.002329 | lncRNA | hg38 |
| 880.1 | | | | | 205 | | | | | | |
| ENST00000564 | 16 | 22302974 | 22309945 | + | POLR3E− | POLR3E | 449 | 2.112213083 | 0.020807 | lncRNA | hg38 |
| 256.1 | | | | | 210 | | | | | | |
| ENST00000514 | 16 | 89686728 | 89691512 | + | CDK10-215 | CDK10 | 474 | 2.008520354 | 0.015893 | lncRNA | hg38 |
| 965.5 | | | | | | | | | | | |
| ENST00000506 | 4 | 52626128 | 52656573 | − | USP46-206 | USP46 | 536 | 1.913483309 | 0.014963 | lncRNA | hg38 |
| 707.1 | | | | | | | | | | | |
| ENST00000556 | 14 | 75278828 | 75279531 | + | FOS-209 | FOS | 596 | 1.89917802 | 4.28E−05 | lncRNA | hg38 |
| 324.2 | | | | | | | | | | | |
| ENST00000470 | 10 | 122932603 | 122952007 | − | C10orf88- | C10orf88 | 675 | 1.780977388 | 0.035682 | lncRNA | hg38 |
| 158.1 | | | | | 203 | | | | | | |
| ENST00000472 | 1 | 78649858 | 78664078 | + | IFI44-206 | IFI44 | 917 | 1.737846893 | 0.007819 | lncRNA | hg38 |
| 152.5 | | | | | | | | | | | |
| ENST00000467 | 1 | 161202349 | 161210696 | + | NDUFS2- | NDUFS2 | 1060 | 1.731979971 | 0.029773 | lncRNA | hg38 |
| 295.5 | | | | | 204 | | | | | | |
| ENST00000476 | 1 | 78620469 | 78641550 | + | IFI44L-208 | IFI44L | 890 | 1.675255629 | 0.002186 | lncRNA | hg38 |
| 876.5 | | | | | | | | | | | |
| ENST00000414 | 20 | 46901143 | 46901726 | − | AL354766.2- | AL354766.2 | 423 | 1.58201463 | 0.00836 | lncRNA | hg38 |
| 085.1 | | | | | 201 | | | | | | |
| ENST00000475 | 21 | 14224375 | 14227384 | + | RBM11-205 | RBM11 | 668 | 1.559878927 | 0.002435 | lncRNA | hg38 |
| 864.1 | | | | | | | | | | | |
| ENST00000434 | 1 | 148290890 | 148297271 | − | LINC01138- | LINC01138 | 1140 | 1.418028197 | 0.001703 | lncRNA | hg38 |
| 245.3 | | | | | 201 | | | | | | |
| ENST00000527 | 11 | 119106942 | 119107758 | − | C2CD2L- | C2CD2L | 569 | 1.408675091 | 0.006371 | lncRNA | hg38 |
| 854.1 | | | | | 203 | | | | | | |
| ENST00000480 | 1 | 85581200 | 85582099 | + | CYR61-202 | CYR61 | 551 | 1.405394963 | 0.000917 | lncRNA | hg38 |
| 413.1 | | | | | | | | | | | |
| ENST00000495 | 1 | 112699624 | 112700722 | + | MOV10-216 | MOV10 | 705 | 1.394988847 | 0.032146 | lncRNA | hg38 |
| 374.5 | | | | | | | | | | | |
| ENST00000645 | 11 | 65423125 | 65426499 | + | NEAT1-207 | NEAT1 | 3300 | 1.335075539 | 0.003829 | lncRNA | hg38 |
| 023.1 | | | | | | | | | | | |
| ENST00000567 | 16 | 56608690 | 56609497 | + | MT2A-205 | MT2A | 416 | 1.325648747 | 0.008549 | lncRNA | hg38 |
| 300.1 | | | | | | | | | | | |
| ENST00000587 | 18 | 47108378 | 47150476 | − | HDHD2-204 | HDHD2 | 788 | 1.324260422 | 0.048842 | lncRNA | hg38 |
| 841.5 | | | | | | | | | | | |
| ENST00000465 | 10 | 86958618 | 86962873 | + | SNCG-203 | SNCG | 641 | 1.314657589 | 0.002155 | lncRNA | hg38 |
| 679.5 | | | | | | | | | | | |
| ENST00000606 | 5 | 93411018 | 93438737 | − | NR2F1- | NR2F1- | 527 | 1.290120222 | 0.003102 | lncRNA | hg38 |
| 188.1 | | | | | AS1-207 | AS1 | | | | | |
| ENST00000497 | X | 106640455 | 106669212 | + | CXorf57- | CXorf57 | 682 | 1.278638922 | 0.018933 | lncRNA | hg38 |
| 124.1 | | | | | 206 | | | | | | |
| ENST00000499 | 11 | 65422774 | 65426457 | + | NEAT1-201 | NEAT1 | 3441 | 1.260273783 | 0.007866 | lncRNA | hg38 |
| 732.3 | | | | | | | | | | | |
| ENST00000587 | 17 | 60083572 | 60088467 | − | WFDC21P- | WFDC21P | 567 | 1.256508214 | 0.007955 | lncRNA | hg38 |
| 298.1 | | | | | 202 | | | | | | |
| ENST00000483 | 10 | 86959375 | 86963258 | + | SNCG-204 | SNCG | 794 | 1.237726865 | 0.000981 | lncRNA | hg38 |
| 064.1 | | | | | | | | | | | |
| ENST00000609 | 7 | 879790 | 886547 | − | AC073957.3- | AC073957.3 | 6758 | 1.222507195 | 1.87E−06 | lncRNA | hg38 |
| 998.1 | | | | | 201 | | | | | | |
| ENST00000487 | 1 | 77979175 | 78016274 | + | DNAJB4- | DNAJB4 | 949 | 1.220029424 | 0.000712 | lncRNA | hg38 |
| 931.1 | | | | | 206 | | | | | | |
| ENST00000565 | 16 | 56617476 | 56618818 | + | MT1L-201 | MT1L | 411 | 1.215439615 | 7.9E−05 | lncRNA | hg38 |
| 768.1 | | | | | | | | | | | |
| ENST00000612 | 11 | 65422804 | 65424404 | + | NEAT1-204 | NEAT1 | 1053 | 1.212262391 | 0.005812 | lncRNA | hg38 |
| 303.2 | | | | | | | | | | | |
| ENST00000587 | 18 | 23573452 | 23576947 | − | NPC1-206 | NPC1 | 590 | 1.199490404 | 0.035479 | lncRNA | hg38 |
| 223.1 | | | | | | | | | | | |
| ENST00000531 | 11 | 67215911 | 67256374 | + | KDM2A- | KDM2A | 4160 | 1.197210843 | 0.044602 | lncRNA | hg38 |
| 696.5 | | | | | 213 | | | | | | |
| ENST00000605 | 1 | 156641666 | 156644887 | − | AL365181.3- | AL365181.3 | 3222 | 1.162752946 | 2.65E−06 | lncRNA | hg38 |
| 886.1 | | | | | 201 | | | | | | |
| ENST00000448 | 1 | 156646507 | 156661424 | − | AL590666.2- | AL590666.2 | 758 | 1.134995997 | 5.86E−05 | lncRNA | hg38 |
| 869.1 | | | | | 201 | | | | | | |
| ENST00000461 | 22 | 31716727 | 31750072 | − | PRR14L- | PRR14L | 718 | 1.093319068 | 0.004039 | lncRNA | hg38 |
| 722.1 | | | | | 206 | | | | | | |
| ENST00000584 | 17 | 82101460 | 82106375 | − | CCDC57- | CCDC57 | 513 | 1.069742202 | 0.00767 | lncRNA | hg38 |
| 717.1 | | | | | 219 | | | | | | |
| ENST00000478 | 1 | 201983375 | 202003420 | + | RNPEP-207 | RNPEP | 1069 | 1.036927696 | 0.037395 | lncRNA | hg38 |
| 617.5 | | | | | | | | | | | |
| ENST00000411 | 10 | 123027534 | 123040657 | + | ACADSB- | ACADSB | 512 | 1.032057823 | 0.049295 | lncRNA | hg38 |
| 816.2 | | | | | 203 | | | | | | |
| ENST00000520 | 5 | 159227715 | 159245127 | + | LINC01932- | LINC01932 | 573 | 1.014164567 | 0.042002 | lncRNA | hg38 |
| 323.1 | | | | | 201 | | | | | | |
| ENST00000462 | 3 | 183287480 | 183298504 | + | B3GNT5- | B3GNT5 | 2748 | 1.008324327 | 4.12E−07 | lncRNA | hg38 |
| 559.1 | | | | | 203 | | | | | | |

As described herein, the compositions and methods may use a biomarker panel comprising two or more genes listed in Tables 1-3. In some embodiments, the expression levels of one or more of these genes may change (e.g., increase or decrease) as induced by a KRAS mutation. In some embodiments, the expression levels of one or more of these genes may increase or decrease as induced by a KRAS mutation. In some embodiments, the expression levels of one or more of these genes may change (e.g., increase or decrease) in one or more specific tissue types (e.g., lung, kidney, and/or pancreas tissues) as induced by a KRAS mutation.

### III. Methods of the Invention

The methods of the invention include measuring and analyzing the expression levels of one or more genes in Tables 1-3 in a biological sample from a subject and diagnosing whether the subject has cancer and/or a KRAS mutation based on the differential expression levels of the genes in the biological sample of the subject compared to the expression levels of the corresponding reference genes in a control sample from a control subject.

In some embodiments, if the gene in the biological sample from the subject displays a differential expression level relative to the corresponding reference gene in the control sample from the control subject, i.e., higher or lower than the expression level of the gene in the control sample by at least 2%, 4%, 6%, 8%, 10%, 20%, 30%, 40%, or 50%, then the subject may have cancer and/or a KRAS mutation. In certain embodiments, the cancer and/or the KRAS mutation may be in a tissue of the subject (e.g., lung).

In some embodiments, the method comprises analyzing the expression level of one or more genes involved in the interferon (IFN) alpha or gamma response. The expression level of one or more genes involved in the IFN alpha or gamma response can increase in response to a KRAS mutation. In other embodiments, the method comprises analyzing the expression level of a gene encoding pattern recognition receptor (PRR). The expression level of the gene encoding the PRR can increase in response to a KRAS mutation. In other embodiments, the method comprises analyzing the expression level of a gene encoding cytosolic RNA sensor RIG-I or MDA5. The expression level of the gene encoding the cytosolic RNA sensor RIG-I or MDA5 can increase in response to a KRAS mutation. In yet other embodiments, the method comprises analyzing the expression level of a gene encoding a KRAB zinc-finger (KZNF) protein. The expression level of a gene encoding a KZNF protein can decrease in response to a KRAS mutation.

As described herein, the methods may further comprise identifying a tissue source (e.g., lung, kidney, or pancreas tissue) of the cancer based on the differential expression levels of the one or more genes in Tables 1-3 in the biological sample compared to the expression levels of the corresponding reference genes in the control sample.

Moreover, once a subject is diagnosed to have cancer based on the differential expression levels of the genes in Tables 1-3 in the biological sample of the subject compared to the expression levels of the corresponding reference genes in the control sample from the control subject, the subject may be administered one or more anticancer agents. In certain embodiments, an anticancer agent can be an inhibitor of a KRAS mutation. In other embodiments, an anticancer agent can be an inhibitor of the gene in Tables 1-3 that is identified to have a differential expression level compared to the corresponding reference level for the gene in the control sample. Examples of inhibitors and examples of anticancer agents are described in detail further herein.

In the methods described herein, in some embodiments, the subject is suspected of having a KRAS mutation, e.g., a KRAS mutation is in a lung, kidney, or pancreas tissue of the subject.

In the methods described herein, in some embodiments, the cancer is a lung cancer (e.g., lung adenocarcinoma). The cancer may be characterized by an oncogenic defect in the RAS pathway. In particular embodiments, the oncogenic defect comprises an activating mutation in KRAS.

### IV. Inhibitors

In some embodiments of the methods described herein, an increased expression level of a gene in Tables 1-3 in a biological sample from a subject compared to a corresponding reference expression level of the same gene in a control sample from a control subject may indicate that the subject has cancer. In some embodiments of the methods described herein, once it is determined that a subject (e.g., a subject suspected of having cancer) has an increased expression level of the gene relative to a control sample, the subject may be administered a therapeutically effective amount of an inhibitor to inhibit the expression level of the gene.

An inhibitor of the gene refers to an agent that inhibits or decreases the expression level and/or the activity of the gene. An inhibitor may inhibits or decreases the transcription of the gene, binds to the gene, and/or inhibits interaction between the gene and another protein or nucleic acid. In some embodiments, an inhibitor may be an inhibitory RNA (e.g., small interfering RNA (siRNA), an antisense RNA, microRNA (miRNA), and short hairpin RNA), an aptamer, an antibody, or a small molecule.

In some embodiments, an inhibitor may be an inhibitory RNA, e.g., small interfering RNA (siRNA), an antisense RNA, microRNA (miRNA), or short hairpin RNA (shRNA). In some embodiments, the inhibitory RNA targets a sequence that is identical or substantially identical (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identical) to a target sequence in the gene. A target sequence in the gene may be a portion of the gene comprising at least 20, at least 30, at least 40, at least 50, at least 60, at least 70, at least 80, at least 90, or at least 100 contiguous nucleotides, e.g., from 20-500, 20-250, 20-100, 50-500, or 50-250 contiguous nucleotides.

In some embodiments of the methods described herein, once it is determined that a subject (e.g., a subject suspected of having cancer) has an increased expression level of one or more genes in Tables 1-3 relative to a control sample, the subject may be administered a therapeutically effective amount of an siRNA that inhibits or decreases the expression level of the gene. An siRNA may be produced from a short hairpin RNA (shRNA). A shRNA is an artificial RNA molecule with a hairpin turn that can be used to silence target gene expression via the siRNA it produces in cells. See, e.g., Fire et. al., Nature 391:806-811, 1998; Elbashir et al., Nature 411:494-498, 2001; Chakraborty et al., Mol Ther Nucleic Acids 8:132-143, 2017; and Bouard et al., Br. J. Pharmacol. 157:153-165, 2009. Expression of shRNA in cells is typically accomplished by delivery of plasmids or through viral or bacterial vectors. Suitable bacterial vectors include but not limited to adeno-associated viruses (AAVs), adenoviruses, and lentiviruses. After the vector has integrated into the host genome, the shRNA is then transcribed in the nucleus by polymerase II or polymerase III (depending on the promoter used). The resulting pre-shRNA is exported from the nucleus, then processed by Dicer and loaded into the RNA-induced silencing complex (RISC). The sense strand is degraded by RISC and the antisense strand directs RISC to an mRNA that has a complementary sequence. A protein called Ago2 in the RISC then cleaves the mRNA, or in some cases, represses translation of the mRNA, leading to its destruction and an eventual reduction in the protein encoded by the mRNA. Thus, the shRNA leads to targeted gene silencing.

In some embodiments, once it is determined that a subject (e.g., a subject suspected of having cancer) has an increased expression level of one or more genes in Tables 1-3 relative to a control sample, the subject may be administered a therapeutically effective amount of an shRNA capable of hybridizing to a portion of the gene. The shRNA may be encoded in a vector. In some embodiments, the vector further comprises appropriate expression control elements known in the art, including, e.g., promoters (e.g., inducible promoters or tissue specific promoters), enhancers, and transcription terminators.

In some embodiments, once it is determined that a subject (e.g., a subject suspected of having cancer) has an increased expression level of one or more genes in Tables 1-3 relative to a control sample, the subject may be administered a therapeutically effective amount of an siRNA capable of hybridizing to a portion of the gene. The siRNA may be encoded in a vector. In some embodiments, the vector further comprises appropriate expression control elements known in the art, including, e.g., promoters (e.g., inducible promoters or tissue specific promoters), enhancers, and transcription terminators.

### V. Detecting Expression Levels

Techniques and methods for measuring the expression levels of genes are available in the art. For example, detection and/or quantification of genes in Tables 1-3 may be accomplished by any one of a number methods or assays employing recombinant DNA or RNA technologies known in the art, including but not limited to, polymerase chain reaction (PCR), single-cell RNA-sequencing, reverse transcription PCR (RT-PCR), microarrays, Northern blot, serial analysis of gene expression (SAGE), immunoassay, hybridization capture, cDNA sequencing, direct RNA sequencing, nanopore sequencing, and mass spectrometry.

In some embodiments, hybridization capture methods may be used for detection and/or quantification of the genes in Tables 1-3. Some examples of hybridization capture methods include, e.g., capture hybridization analysis of RNA targets (CHART), chromatin isolation by RNA purification (ChIRP), and RNA affinity purification (RAP). In general, cells and tissues expressing the RNA of interest can be cross-linked and solubilized by shearing. The RNA of interest can then be enriched using rationally designed biotin tagged antisense oligonucleotides. The captured RNA complexes can then be rinsed and eluted. The eluted material can be analyzed for the molecules of interest. The associated RNAs are commonly analyzed with qPCR or high throughput sequencing, and the recovered proteins can be analyzed with Western blots or mass spectrometry. General techniques for performing hybridization capture methods are described in the art and can be found in, e.g., Machyna and Simon, Briefings in Functional Genomics 17(2):96-103, 2018, which is incorporated herein by reference in its entirety. Further, Li et al, JCI Insight. 3(7):e98942, 2018 also describes methods of studying RNA (e.g., extracellular RNA) and is incorporated herein by reference in its entirety.

In some embodiments, microarrays may be used to measure the expression levels of the genes. An advantage of microarray analysis is that the expression of each of the genes can be measured simultaneously, and microarrays can be specifically designed to provide a diagnostic expression profile for a particular disease or condition (e.g., cancer). Microarrays may be prepared by selecting probes which comprise a polynucleotide sequence, and then immobilizing such probes to a solid support or surface. For example, the probes may comprise DNA sequences, RNA sequences, or copolymer sequences of DNA and RNA. The polynucleotide sequences of the probes may also comprise DNA and/or RNA analogues, or combinations thereof. For example, the polynucleotide sequences of the probes may be full or partial fragments of genomic nucleic acids. The polynucleotide sequences of the probes may also be synthesized nucleotide sequences, such as synthetic oligonucleotide sequences. Probes may be immobilized to a solid support which may be either porous or non-porous. For example, the probes may be polynucleotide sequences which are attached to a nitrocellulose or nylon membrane or filter covalently at either the 3′ or the 5′ end of the polynucleotide. Such hybridization probes are well-known in the art (see, e.g., Sambrook, et al., Molecular Cloning: A Laboratory Manual (3rd Ed., 2001). In one embodiment, a microarray may include a support or surface with an ordered array of binding (e.g., hybridization) sites or “probes” each representing one of the genes described herein. More specifically, each probe of the array may be located at a known, predetermined position on the solid support such that the identity (i.e., the sequence) of each probe can be determined from its position in the array (i.e., on the support or surface). Each probe may be covalently attached to the solid support at a single site.

Quantitative reverse transcriptase PCR (qRT-PCR) can also be used to determine the expression profiles of the genes. The first step in gene expression profiling by RT-PCR is the reverse transcription of the RNA template into cDNA, followed by its exponential amplification in a PCR reaction. The two most commonly used reverse transcriptases are avilo myeloblastosis virus reverse transcriptase (AMY-RT) and Moloney murine leukemia virus reverse transcriptase (MLVRT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, CA, USA), following the manufacturer's instructions. The derived cDNA can then be used as a template in the subsequent PCR reaction. Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TAQMAN PCR typically utilizes the 5′-nuclease activity of Taq polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, may be designed to detect nucleotide sequence located between the two PCR primers. The probe is non-extendible by Taq DNA polymerase enzyme, and may be labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

Serial Analysis Gene Expression (SAGE) can also be used to determine RNA expression level. SAGE analysis does not require a special device for detection, and may be used for simultaneously detecting the expression of a large number of transcription products. First, RNA is extracted, converted into cDNA using a biotinylated oligo (dT) primer, and treated with a four-base recognizing restriction enzyme (Anchoring Enzyme: AE) resulting in AE-treated fragments containing a biotin group at their 3′ terminus. Next, the AE-treated fragments are incubated with streptavidin for binding. The bound cDNA is divided into two fractions, and each fraction is then linked to a different double-stranded oligonucleotide adapter (linker) A or B. These linkers are composed of: (1) a protruding single strand portion having a sequence complementary to the sequence of the protruding portion formed by the action of the anchoring enzyme, (2) a 5′ nucleotide recognizing sequence of the IIS-type restriction enzyme (cleaves at a predetermined location no more than 20 bp away from the recognition site) serving as a tagging enzyme (TE), and (3) an additional sequence of sufficient length for constructing a PCR-specific primer. The linker-linked cDNA is cleaved using the tagging enzyme, and only the linker-linked cDNA sequence portion remains, which is present in the form of a short-strand sequence tag. Next, pools of short-strand sequence tags from the two different types of linkers are linked to each other, followed by PCR amplification using primers specific to linkers A and B. As a result, the amplification product is obtained as a mixture comprising myriad sequences of two adjacent sequence tags (ditags) bound to linkers A and B. The amplification product is treated with the anchoring enzyme, and the free ditag portions are linked into strands in a standard linkage reaction. The amplification product is then cloned. Determination of the clone's nucleotide sequence can be used to obtain a readout of consecutive ditags of constant length. The presence of the gene corresponding to each tag can then be identified from the nucleotide sequence of the clone and information on the sequence tags.

One of skill in the art, when provided with the set of genes in Tables 1-3 to be identified and quantified, will be capable of selecting the appropriate assay for performing the methods disclosed herein.

### VI. Anticancer Agents

In methods described herein, a subject may be administered one or more anticancer agents alone or in combination with one or more inhibitors that inhibit the expression levels of one or more genes in Tables 1-3. An anticancer agent may be a cytotoxic agent, a chemotherapeutic agent, or an immunosuppressive agent. An anticancer agent may be a natural or synthetic agent. In some embodiments, an anticancer agent may be capable of treating cancer, activating immune response, and/or reducing tumor load. In some embodiments, an anticancer agent may inhibit the proliferation of and/or kill cancer cells. An anticancer agent may be a small molecule, a peptide, or a protein. In some embodiments, an anticancer agent may be an agent that inhibits and/or down regulates the activity of a protein that prevents immune cell activation or a protein that exerts immunosuppressive effects.

Examples of anticancer agents include, but are not limited to, alkylating agents such as thiotepa and cyclosphosphamide (CYTOXAN®); alkyl sulfonates such as busulfan, improsulfan and piposulfan; aziridines such as benzodopa, carboquone, meturedopa, and uredepa; ethylenimines and methylamelamines including altretamine, triethylenemelamine, triethyl lenephosphoramide, triethyl lenethiophosphoramide and trimethylmelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopoletin, and 9)-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Nicolaou et al. Angew. Chem Intl. Ed. Engl., 33: 183-186 (1994)); CDP323, an oral alpha-4 integrin inhibitor; dynemicin, including dynemicin A; an esperamicin; neocarzinostatin chromophore and related chromoprotein enediyne antibiotic chromophores), aclacinomysins, actinomycin, authramycin, azaserine, bleomycin, cactinomycin, carabicin, caminomycin, carzinophilin, chromomycins, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®), liposomal doxorubicin TLC D-99 (MYOCET®), peglylated liposomal doxorubicin (CAELYX®), and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); combretastatin; folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, 5-azacytidine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2′-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoid, e.g., paclitaxel (TAXOL®, Bristol-Myers Squibb Oncology, Princeton, N.J.), albumin-engineered nanoparticle formulation of paclitaxel (ABRAXANE™), and docetaxel (TAXOTERE®, Rhome-Poulene Rorer, Antony, France); chloranbucil; 6-thioguanine; mercaptopurine; methotrexate; platinum agents such as cisplatin, oxaliplatin (e.g., ELOXATIN®), and carboplatin; vincas, which prevent tubulin polymerization from forming microtubules, including vinblastine (VELBAN®), vincristine (ONCOVIN®), vindesine (ELDISINE®, FILDESIN®), and vinorelbine (NAVELBINE®); etoposide (VP-16); ifosfamide; mitoxantrone; leucovorin; novantrone; edatrexate; daunomycin; aminopterin; ibandronate; topoisomerase inhibitor RFS 2000; difluoromethylornithine (DMFO); retinoids such as retinoic acid, including bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R) (e.g., erlotinib (Tarceva™)); and VEGF-A that reduce cell proliferation; vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); BAY439006 (sorafenib; Bayer); SU-11248 (sunitinib, SUTENT®, Pfizer); perifosine, COX-2 inhibitor (e.g. celecoxib or etoricoxib), proteosome inhibitor (e.g. PS341); bortezomib (VELCADE®); CCI-779; tipifarnib (R11577); orafenib, ABT510); Bcl-2 inhibitor such as oblimersen sodium (GENASENSE®); pixantrone; EGFR inhibitors; tyrosine kinase inhibitors; serine-threonine kinase inhibitors such as rapamycin (sirolimus, RAPAMUNE®); farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

In some embodiments, an anticancer agent is cisplatin, carboplatin, oxaliplatin, bleomycin, mitomycin C, calicheamicins, maytansinoids, doxorubicin, idarubicin, daunorubicin, epirubicin, busulfan, carmustine, lomustine, semustine, methotrexate, 6-mercaptopurine, fludarabine, 5-azacytidine, pentostatin, cytarabine, gemcitabine, 5-fluorouracil, hydroxyurea, etoposide, teniposide, topotecan, irinotecan, chlorambucil, cyclophosphamide, ifosfamide, melphalan, bortezomib, vincristine, vinblastine, vinorelbine, paclitaxel, or docetaxel.

### Chemotherapeutic Agent

In some embodiments, the anticancer agent is a chemotherapeutic agent. In some embodiments, chemotherapeutic agents may kill cancer cells or inhibit cancer cell growth. Chemotherapeutic agents may function in a non-specific manner, for example, inhibiting the process of cell division known as mitosis. Examples of chemotherapeutic agents include, but are not limited to, antimicrotubule agents (e.g., taxanes and vinca alkaloids), topoisomerase inhibitors and antimetabolites (e.g., nucleoside analogs acting as such, for example, Gemcitabine), mitotic inhibitors, alkylating agents, antimetabolites, antitumor antibiotics, mitotic inhibitors, anthracyclines, intercalating agents, agents capable of interfering with a signal transduction pathway, agents that promote apoptosis, proteosome inhibitors, and alike.

Alkylating agents are most active in the resting phase of the cell. These types of drugs are cell-cycle non-specific. Exemplary alkylating agents include, but are not limited to, nitrogen mustards, ethylenimine derivatives, alkyl sulfonates, nitrosoureas and triazenes); uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®), Neosar®, Clafen®, Endoxan® Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BICNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

Antitumor antibiotics are chemotherapeutic agents obtained from natural products produced by species of the soil fungus, e.g., Streptomyces. These drugs act during multiple phases of the cell cycle and are considered cell-cycle specific. There are several types of antitumor antibiotics, including but are not limited to anthracyclines (e.g., Doxorubicin, Daunorubicin, Epirubicin, Mitoxantrone, and Idarubicin), chromomycins (e.g., Dactinomycin and Plicamycin), mitomycin, and bleomycin.

Antimetabolites are types of chemotherapeutic agents that are cell-cycle specific. When cells incorporate these antimetabolite substances into the cellular metabolism, they are unable to divide. This class of chemotherapeutic agents include folic acid antagonists such as Methotrexate; pyrimidine antagonists such as 5-Fluorouracil, Foxuridine, Cytarabine, Capecitabine, and Gemcitabine; purine antagonists such as 6-Mercaptopurine and 6-Thioguanine; Adenosine deaminase inhibitors such as Cladribine, Fludarabine, Nelarabine and Pentostatin.

Exemplary anthracyclines that can be used include, e.g., doxorubicin (Adriamycin® and Rubex®); Bleomycin (Lenoxane®); Daunorubicin (dauorubicin hydrochloride, daunomycin, and rubidomycin hydrochloride, Cerubidine®); Daunorubicin liposomal (daunorubicin citrate liposome, DaunoXome®); Mitoxantrone (DHAD, Novantrone®); Epirubicin (Ellence); Idarubicin (Idamycin®, Idamycin PFS®); Mitomycin C (Mutamycin®); Geldanamycin; Herbimycin; Ravidomycin; and Desacetylravidomycin.

Antimicrotubule agents include vinca alkaloids and taxanes. Exemplary vinca alkaloids include, but are not limited to, vinorelbine tartrate (Navelbine®), Vincristine (Oncovin®), and Vindesine (Eldisine®); vinblastine (also known as vinblastine sulfate, vincaleukoblastine and VLB, Alkaban-AQ® and Velban®); and vinorelbine (Navelbine®). Exemplary taxanes that can be used include, but are not limited to paclitaxel and docetaxel. Non-limiting examples of paclitaxel agents include nanoparticle albumin-bound paclitaxel (ABRAXANE, marketed by Abraxis Bioscience), docosahexaenoic acid bound-paclitaxel (DHA-paclitaxel. Taxoprexin, marketed by Protarga), polyglutamate bound-paclitaxel (PG-paclitaxel, paclitaxel poliglumex, CT-2103, XYOTAX, marketed by Cell Therapeutic), the tumor-activated prodrug (TAP), ANG105 (Angiopep-2 bound to three molecules of paclitaxel, marketed by ImmunoGen), paclitaxel-EC-1 (paclitaxel bound to the erbB2-recognizing peptide EC-1; see Li et al., Biopolymers (2007) 87:225-230), and glucose-conjugated paclitaxel (e.g., 2′-paclitaxel methyl 2-glucopyranosyl succinate, see Liu et al., Bioorganic & Medicinal Chemistry Letters (2007) 17:617-620).

Exemplary proteosome inhibitors that can be used include, but are not limited to, Bortezomib (Velcade®); Carfilzomib (PX-171-007, (S)-4-Methyl-N—((S)-1-(((S)-4-methyl-1-((R)-2-methyloxiran-2-yl)-1-oxope-ntan-2-yl)amino)-1-oxo-3-phenylpropan-2-yl)-2-((S)-2-(2-morpholinoacetamid-o)-4-phenylbutanamido)-pentanamide); marizomib (NPI-0052); ixazomib citrate (MLN-9708); delanzomib (CEP-18770); and O-Methyl-N-[(2-methyl-5-thiazolyl)carbonyl]-L-seryl-O-methyl-N-[(1S)-2-[(-2R)-2-methyl-2-oxiranyl]-2-oxo-1-(phenylmethyl)ethyl]-L-serinamide (ONX-0912).

In some embodiments, the chemotherapeutic agent is selected from the group consisting of chlorambucil, cyclophosphamide, ifosfamide, melphalan, streptozocin, carmustine, lomustine, bendamustine, uramustine, estramustine, carmustine, nimustine, ranimustine, mannosulfan busulfan, dacarbazine, temozolomide, thiotepa, altretamine, 5-fluorouracil (5-FU), 6-mercaptopurine (6-MP), capecitabine, cytarabine, floxuridine, fludarabine, gemcitabine, hydroxyurea, methotrexate, pemetrexed, daunorubicin, doxorubicin, epirubicin, idarubicin, SN-38, ARC, NPC, campothecin, topotecan, 9-nitrocamptothecin, 9-aminocamptothecin, rubifen, gimatecan, diflomotecan, BN80927, DX-895 If, MAG-CPT, amsacrine, etoposide, etoposide phosphate, teniposide, doxorubicin, paclitaxel, docetaxel, gemcitabine, accatin III, 10-deacetyltaxol, 7-xylosyl-10-deacetyltaxol, cephalomannine, 10-deacetyl-7-epitaxol, 7-epitaxol, 10-deacetylbaccatin III, 10-deacetyl cephalomannine, gemcitabine, Irinotecan, albumin-bound paclitaxel, Oxaliplatin, Capecitabine, Cisplatin, docetaxel, irinotecan liposome, and etoposide, and combinations thereof.

In certain embodiments, the chemotherapeutic agent is administered at a dose and a schedule that may be guided by doses and schedules approved by the U.S. Food and Drug Administration (FDA) or other regulatory body, subject to empirical optimization.

In still further embodiments, more than one chemotherapeutic agent may be administered simultaneously, or sequentially in any order during the entire or portions of the treatment period. The two agents may be administered following the same or different dosing regimens.

### EXAMPLES

### Example 1—Materials and Methods

### Cell Lines

The AALE stable cell lines pBABE-mCherry Puro (control) and pBABE-FLAG-KRAS(G12) Zeo (mutant KRAS) were generated using retroviral transduction, followed by selection in puromycin of zeocin, respectively, 2 days post-infection. Both lines were cultured in SABM Basal Medium (Lonza SABM basal medium) with added supplements and growth factors (Lonza SAGM SingleQuot Kit Suppl. & Growth Factors). AALE cell lines were maintained using Lonza's Reagent Pack subculture reagents. The HA1E cell lines were generated using lentiviral transduction (pLX317) to generate control and mutant HA1E pLX317-KRAS(G12) stable cell lines using puromycin selection, and cells were cultured in MEM-alpha (Invitrogen) with 10% FBS (Sigma) and 1% penicillin/streptomycin (Gibco). All cell lines tested negative for mycoplasma.

siRNA Knockdowns

AALEs were seeded at 1×10⁶ cells per well of a 6-well plate in complete growth medium, then reverse transfected with 30 pmol siRNA using RNAiMAX lipofectamine according to manufacturer's protocol. Cells were grown for 3 days in transfection medium under standard culture conditions and then harvested for RNA isolation and qPCR as previously described.

### Cell Viability Assay

2×10⁴ cells were subtracted from each siRNA transfection well at the time of transfection and seeded into individual wells of an ultra-low adhesion 96-well plate. The cells were grown in standard culture conditions for 4 days. They were then harvested, and ATP production was measured using the Cell TiterGLO Luminescent Cell Viability Assay (Promega) following the manufacturer's protocol. Luminescence was measured on a Perkin Elmer VICTOR light 1420 Luminescence Counter.

### RNA Isolation & Purification

For AALE cell lines, bulk RNA was isolated from cells using Quick-RNA MiniPrep kit (Zymogen). All RNA was quantified via NanoDrop-8000 Spectrophotometer. For HA1E cell lines, bulk RNA was isolated using RNeasy Mini Kit (Qiagen) and quantified via Qubit RNA BR assay kit (Thermo).

qPCR

cDNA was transcribed from lug RNA using iScript cDNA Synthesis Kit (Bio-Rad) according to manufacturer protocol. cDNA was diluted 1:6 and run with iTaq Universal SYBR Green Supermix (Bio-Rad) on ViiA 7 Real-Time PCR System according to manufacturer protocol. Cycle Threshold (CT) values were converted using Standard analysis. Values obtained for target genes were normalized to HPRT.

### Library Preparation for Bulk RNAseq

For AALE cell lines, lug of total RNA was used as input for the TruSeq Stranded mRNA Sample Prep Kit (Illumina) according to manufacturer protocol. Library quality was determined through the High Sensitivity DNA Kit on a Bioanalyzer 2100 (Agilent Technologies). Multiplexed libraries were sequenced as HiSeq400 100PE runs. For HA1E cell lines, lug of total RNA was used for mRNA enrichment with Dynabeads mRNA DIRECT kit (Thermo). First strand cDNA was generated with AffinityScript Multiple Temperature reverse transcriptase with oligo dT primers. Second strand cDNA was generated with mRNA Second Strand Synthesis Module (New England Biolab). DNA was cleaned up with Agencourt AMPure XP beads twice. Qubit dsDNA High Sensitivity Assay was used for concentration measurement. 1 ng of dsDNA was further subjected to library preparation with Nextera XT DNA sample prep kit (Illumina) per manufacturer instructions. Library size distribution was confirmed with Bioanalyzer (Agilent). Multiplexed libraries were sequenced as NextSeq500 75PE runs.

### Library Preparation for Single Cell RNAseq

For single cell RNAseq, 1×10⁶ cells were harvested and re-suspended in 1 mL 1×PBS/0.04% BSA (1000 cells/ul) according to the cell preparation guidelines in the 10× Genomics Chromium Single Cell 3′ Reagent Kit User Guide. GEMs were generated from an input of 3,500 cells. We used the 10× Genomics Chromium Single Cell 3′ Reagent Kits version 2 for both the GEM generation and subsequent library preparation and followed the manufacturer's reagent kit protocol. Quantification of all RNAseq libraries was performed by QB3 at UC Berkeley. RNAseq libraries were sequenced as HiSeq4000 100PE runs.

### Statistical Analysis

All quantitative data for functional assays has been reported as means±standard deviation. Statistical significance for these was calculated using a t-test and p-values<0.05 were considered significant.

### RNA-seq Pseudoalignment and Quantification

All fastq files were trimmed with Trimmomatic 2 (0.38) [ ] using the Illumina NextSeq PE adapters. The resulting trimmed files were assessed with FastQC [ ] and then passed through the following analytical pipeline:

Salmon (0.14.1): pseudoalignment of RNA-seq reads performed with Salmon [ ] using the following arguments:
-validateMappings -rangeFactorizationBins 4 -gcBias -numBootstraps 10
using an index created from the GENCODE version 29 transcriptome fasta file using standard arguments.

Sleuth (0.30.0): transcript differential expression was performed using Sleuth [ ] and Wasabi (1.0.1) to convert the Salmon output into the proper format. Upon completion, the transcripts with q-values below 0.05 in the likelihood-ratio test were used to filter salmon output from which log 2fc was manually calculated and paired to the sleuth output.

DESeq2 (1.24.0): Salmon output was imported into a DESeq object using tximport [ ] and differential expression analysis was performed with standard arguments.

### Transposable Element Content Analysis

Exon and 5′/3′ UTR Overlap: a whole genome .gtf file was downloaded from the UCSC genome browser Table browser utility. This file was parsed and merged with the GENCODE v.29 reference transcriptome. This modified .gtf (now a .bed file) was passed to bedtools [ ] where the overlap function was used with the following arguments:
a modified.gtf.bed -b all.ucsc.rmsk.genes.bed -wao -s>retained.overlap.bed
alongside a whole genome .gtf retrieved as described above except generated from the repeat-masked browser track. The resulting overlapped bed file was processed and visualized using custom R scripts.

Differential Expression: Differential transcript abundance was determined using the Salmon and Sleuth procedures described above provided with a custom index comprising both the GENCODE version 29 transcripts and all transcripts extracted from the Hammel lab GTF file as described in the single cell procedures. Sleuth output was filtered and visualized using R and Tidyverse.

### Zinc Finger Protein Analysis

ChIP-exo data and supplementary information were extracted from supplementary data provided by Imbeault et al [ ]. ZNF genes were cross referenced with DESeq2 and RepeatMasker outputs to extract relevant differential expression data of ZNF proteins and Transposable Element transcripts using R. RepeatMasker output from promoter analyses was cross referenced with ChIP-exo target data to identify potential regulatory targets of differentially expressed KZNFs. Only KZNF targets with ‘score’ [see Imbeault et al]>=75 were kept for analysis. Analysis of all data was performed and visualized in R using custom scripts.

### Gene Set Enrichment Analysis

Genes determined to be significantly differentially expressed in DESeq2 output were first ‘pre-ranked’ in R by the following metric:

Score metric=sin(log 2FoldChange)*−log₁₀(p-value)

The resulting ranked files objects were processed using the R package fgsea [ ] alongside gene set files downloaded from msigdb [ ] using the R package msigdbr [ ]. Additional code was written for select vizualizations.

### Gene Ontology Analysis

Upregulated gene names were extracted from DESeq2 output using bash command line tools. Name lists were pasted into the Gene Ontology Consortium's Enrichment Analysis tool powered by PANTHER. Output data was exported as .txt files and parsed using bash command line tools. Parsed data was visualized using custom R scripts.

### Single Cell Analysis

10× Processing: Single cell output data was processed using 10× pipeline CellRanger [The mkfastq functionality was used to generate fastq files for further downstream analysis. Output was also aggregated and quantified using the aggr and count functionalities, respectively. This output was visualized using the 10× Loupe browser.

Downstream Analysis: fastq files generated above were passed to Salmn alevin [ ] with the following arguments:
-libtype A -chromium -dumpCsvCounts -p 16.
alevin was used to psuedoalign the libraries to both the GENCODE v.29 reference transcriptome as well as a composite transcriptome reference built by combining the GENCODE v.29 reference with one built from the GRCh38_rmsk_TE.gtf hosted by the Hammel lab. A salmon index was built from this reference with standard arguments. These alevin output matrices were imported into R using tximport. GSEA/cluster correlations were calculated using the R corr( ) function. Normalization and clustering were performed with Seurat [ ] and additional code was written to handle select visualizations.

### TCGA ZNF Analysis

TCGA-LUAD and GTEX lung phenotype and normalized count data were downloaded from the UCSC Xena browser TOIL data repository. The files were combined and patients were grouped by their KRAS mutation status and identity. These data were compared to and visualized alongside of data generated from our analysis using custom R code. Significance was determined with a one-way t test implemented in the R t.test( ) function.

### Example 2—Transcriptome Analysis of Transformed Human Lung Epithelial (AALE) Cells

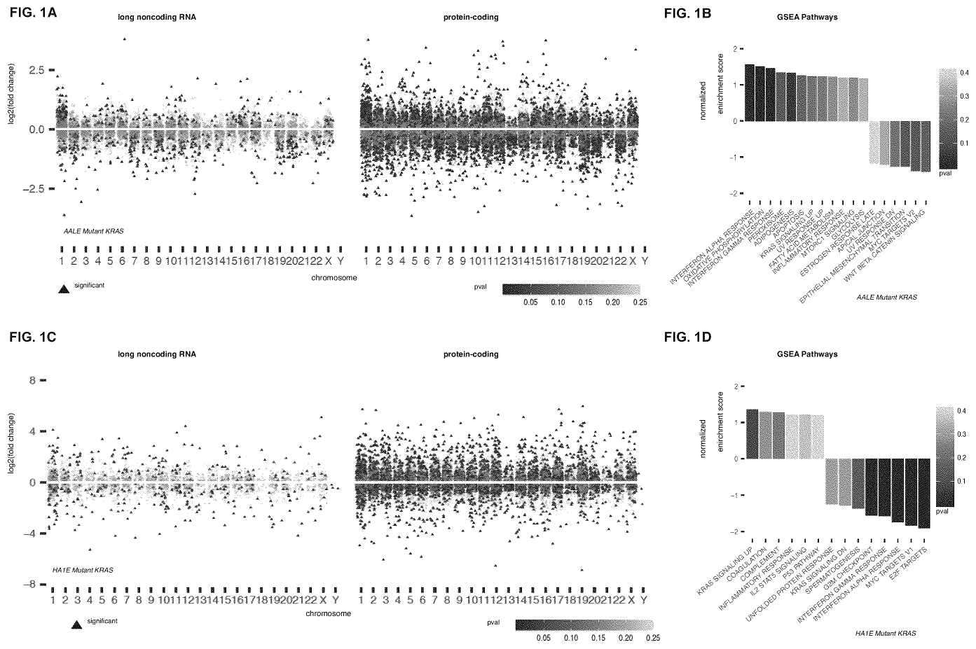

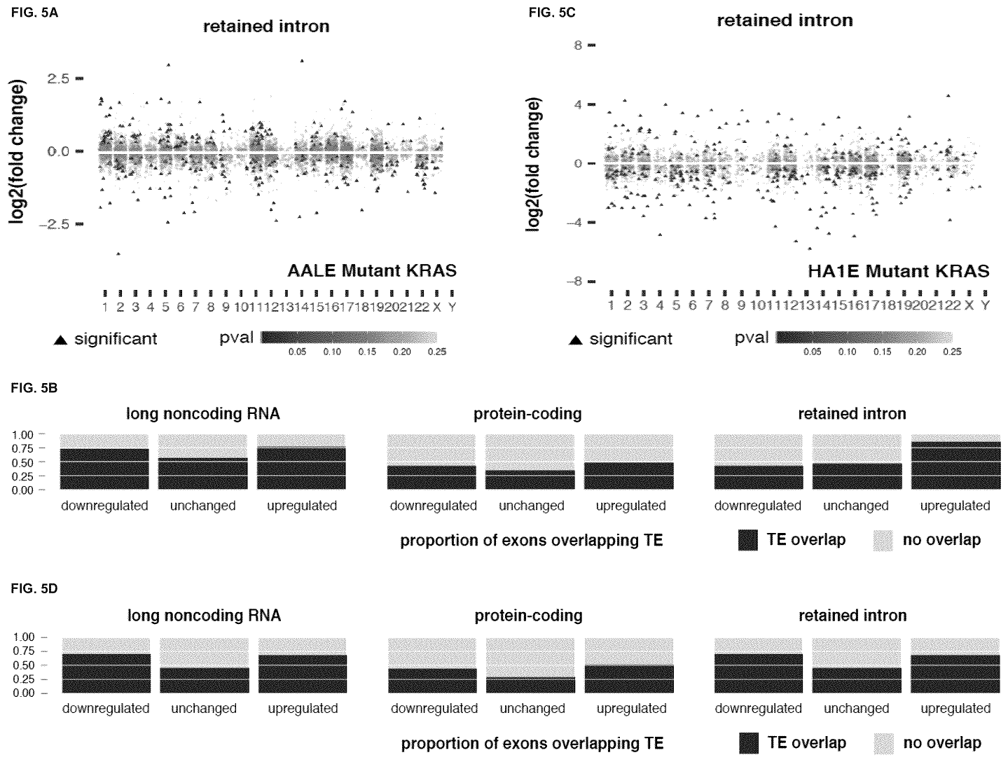

The transcriptomes of AALE cells transduced with control vector and the transcriptomes of AALE cells transduced by mutant KRAS were compared and analyzed. Hundreds of lncRNAs were upregulated (n=279) or downregulated (n=409) by oncogenic RAS signaling, as well as many protein-coding mRNAs (n=4323 up, n=4711 down) (FIG. 1A) and transcripts with retained introns (n=165 up, n=195) (FIG. 5A), revealing the broad extent to which mutant KRAS reprograms the coding and noncoding transcriptome. Compared to transcripts that were expressed but unchanged in the mutant KRAS versus control AALEs, a larger proportion of upregulated or downregulated lncRNAs and protein-coding mRNAs were comprised of TE sequences, while upregulated intron-retaining transcripts were also enriched for TEs (FIG. 5B), suggesting that TE sequence-containing loci in the genome are preferentially misregulated during malignant transformation.

To explore the biological pathways that are perturbed by oncogenic RAS signaling, we performed gene set enrichment analysis (GSEA) (11) using genes that were differentially expressed in our mutant KRAS AALE cells. GSEA revealed that the most significantly enriched pathway was the interferon (IFN) alpha response, while the third most enriched pathway was IFN gamma response (FIG. 1B). These results indicate that mutant KRAS activates an innate immune response in transformed AALEs.

### Example 3—Mutant RAS-Mediated IFN Response

We then investigated whether this mutant RAS-mediated IFN response was specific to lung cells or if unrelated cell types responded similarly. We performed RNA-seq on human embryonic kidney cells (HA1E) that were primed for oncogenic RAS-driven transformation (12) and analyzed how mutant KRAS altered their transcriptomes. We also observed that hundreds of lncRNAs were upregulated (n=165) or downregulated (n=223), along with protein-coding mRNAs (n=2635 up, n=2639 down) (FIG. 1C) and retained-intron transcripts (n=119 up, n=237 down) (FIG. 5C), similar to what we found using mutant KRAS AALE cells. Moreover, differentially expressed RNAs were again enriched for TE sequences (FIG. 5D). When we performed GSEA, however, there was no enrichment for any IFN pathways in mutant KRAS-transformed HA1E cells, even though they were most significantly enriched for upregulated KRAS signaling (FIG. 1D). We found that both IFN gamma and IFN alpha response pathways were among the most significantly decreased gene sets (FIG. 1D), highlighting the tissue-specific differences in how the transcriptome is remodeled by mutant KRAS.

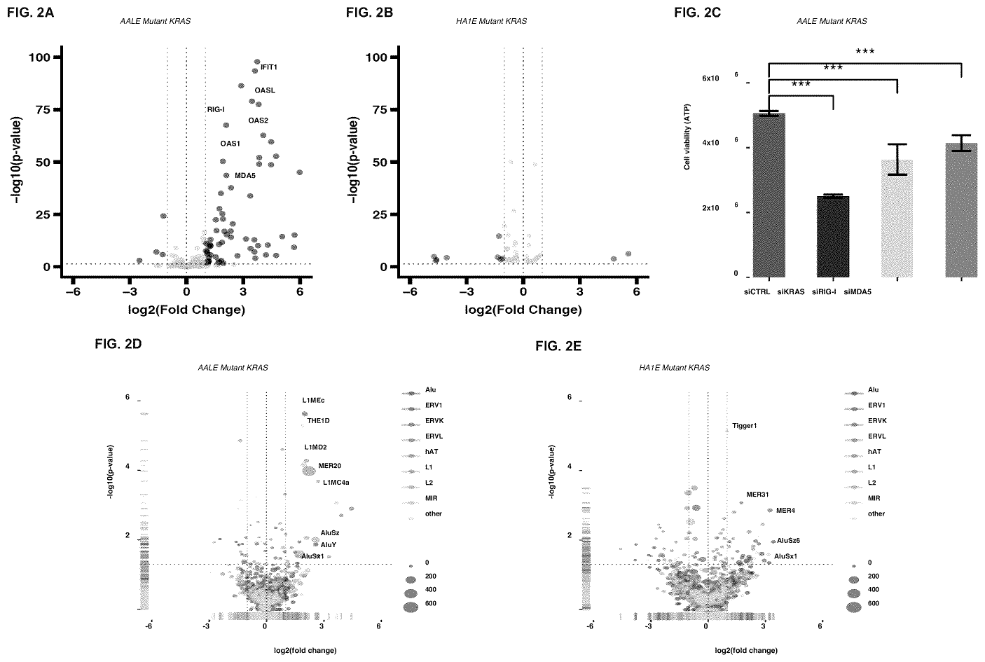

To further elucidate the interferon response in mutant KRAS AALE cells, we compared the expression patterns of differentially expressed IFN-stimulated genes in transformed AALEs and HA1E cells. AALEs with oncogenic RAS signaling upregulated the expression of pattern recognition receptors (PRR) and cytosolic RNA sensors RIG-I and MDA5 (FIG. 2A) (13), while mutant KRAS HA1E cells showed no significant changes in their expression (FIG. 2B). To determine the functional significance of PRR upregulation in the context of RAS-driven cellular transformation, we next performed knockdown studies of RIG-I and MDA5 in mutant KRAS AALE cells. RNA interference-mediated knockdown of KRAS, RIG-I, or MDA5 all resulted in significant loss of cell viability (FIG. 2C), revealing the requirement for heightened levels of RIG-I and MDA5 expression in transformed AALE cells.

### Example 4—Molecular Basis for IFN Pathway Activation in Mutant KRAS AALE Cells

We next investigated the molecular basis for IFN pathway activation in mutant KRAS AALE cells by analyzing the abundance of TE-derived noncoding RNAs, which induce an IFN response in cancer cells when aberrantly expressed (14, 15). The LINE-1 elements L1MEc, L1MD2, and L1MC4a, the ERVL-MaLR element THE1D, and the hAT-Charlie element MER20) were all significantly upregulated in mutant KRAS AALE cells (FIG. 2D) but not in mutant KRAS HA1E cells (FIG. 2E), suggesting that oncogenic KRAS signaling induces an IFN response in transformed lung cells through a tissue-specific set of TE-derived noncoding RNAs.

### Example 5—Single-Cell RNA-Seq

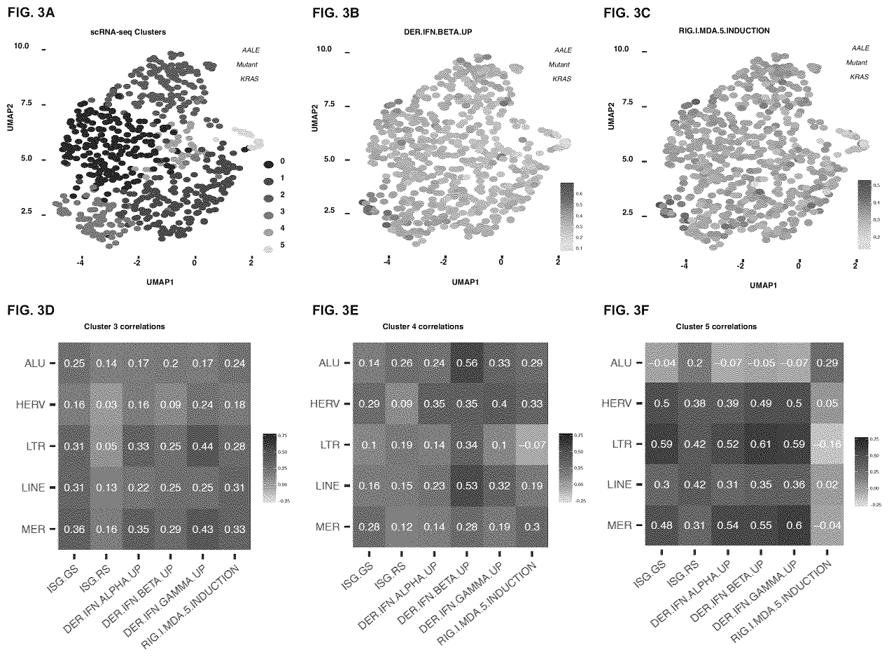

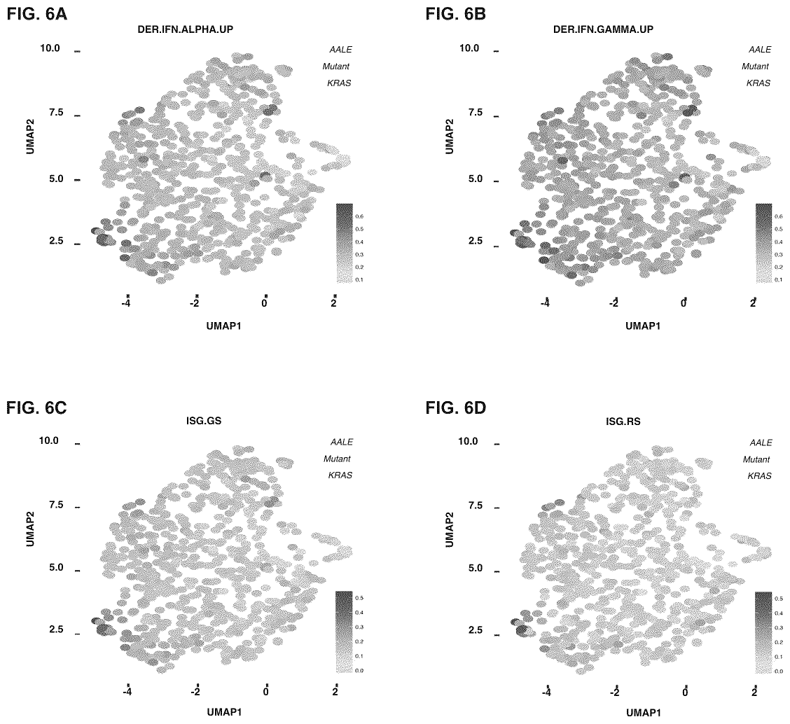

To further characterize the nature of the IFN response in mutant KRAS AALEs, we performed single-cell RNA-seq (scRNA-seq) (n=1503 cells) (FIG. 3A), which revealed that the IFN beta (FIG. 3B), alpha and gamma (FIGS. 6A and 6B) gene signatures were heterogeneously activated in KRAS-transformed AALEs, with a small fraction of individual cells exhibiting very high expression levels of each IFN gene signature. We then analyzed the scRNA-seq data using a RIG-I/MDA5 induction gene signature, which showed that a large fraction of individual cells within this population displayed prominent levels of this PRR signature (FIG. 3C).

We then examined which TE RNAs might be involved in IFN-stimulated gene expression by analyzing scRNA-seq clusters (FIG. 3A) for correlation between TE RNA expression and IFN gene signatures (16). LINE and MER elements were the most highly correlated TE classes with the IFN gamma gene signature in cluster 3 (FIG. 3D), while Alu and LINE elements were highly correlated with the IFN beta gene signature in cluster 4 (FIG. 3E). Cluster 5 showed the strongest correlations between various TE classes and IFN gene signatures, with LTR elements being most highly correlated with the IFN beta gene signature (FIG. 3F). These single cell analyses show that diverse classes of TE-derived noncoding RNAs are likely to induce IFN-related genes in different subsets of mutant KRAS-transformed cells.

### Example 6—Role of KRAB Zinc-Finger Proteins (KZNFs) in TE Silencing

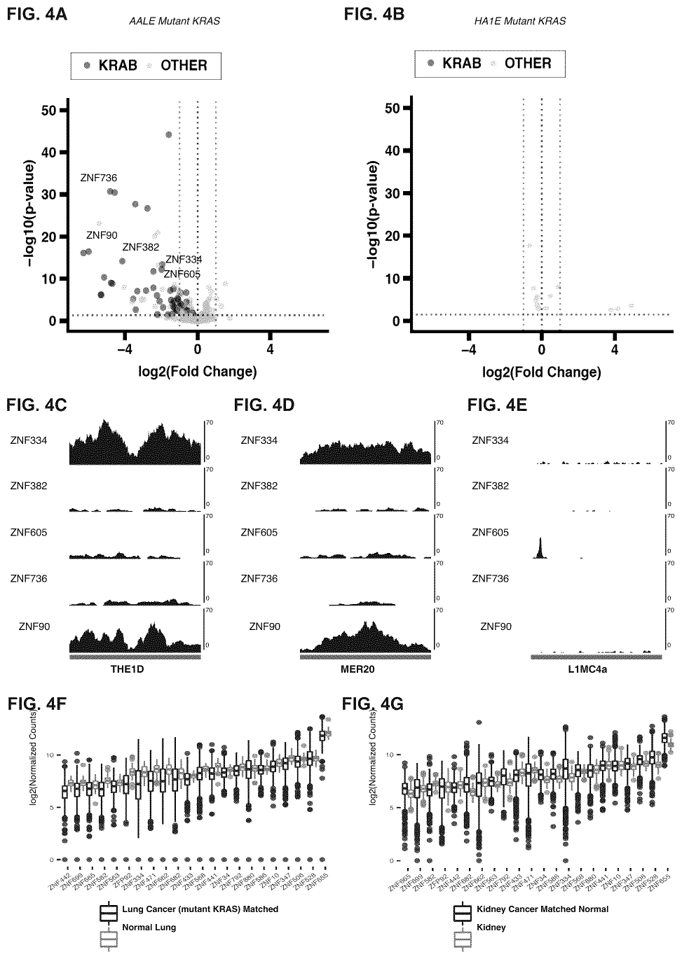

Given the known roles of KRAB zinc-finger proteins (KZNFs) in TE silencing, we examined whether KZNFs were involved in TE regulation in mutant KRAS AALEs. When we examined the differential expression of KZNFs in mutant KRAS AALEs, we observed a broad and significant downregulation of repressive KRAB domain-containing zinc-finger proteins (FIG. 4A). In the mutant KRAS HA1E cells, however, no KZNFs were differentially expressed (FIG. 4B). We then analyzed KZNF chromatin immunoprecipitation sequencing (ChIP-seq) data (17) using a newly developed University of California Santa Cruz (UCSC) Repeat Browser platform. We found that several of the significantly downregulated KZNFs in mutant KRAS AALEs bind to the consensus TE sequences of THE1D (FIG. 4C), MER20) (FIG. 4D), and L1MC4a (FIG. 4E) elements, all of which are specifically and significantly upregulated in mutant KRAS AALEs (FIG. 2D). This suggests that suppression of these KZNFs via oncogenic RAS signaling leads to de-repression of TE-derived noncoding RNAs during cellular transformation. This model is supported by broad and significant downregulation of these same KNZFs in mutant KRAS-driven lung adenocarcinomas (FIG. 4F) but not in kidney cancers (FIG. 4G).

Collectively, our findings illustrate the tissue-specific impact of oncogenic RAS signaling on the noncoding transcriptome. These conclusions are based on deeply sequencing and analyzing the transcriptomes of mutant KRAS-transformed cells at both the population and single-cell levels, building on previous work identifying noncoding RNAs that are coordinately regulated with RAS signaling genes in individual cells (8). The molecular basis for the IFN response we observe in mutant KRAS AALE cells is different from TE-induced IFN responses in cancer cells treated with DNA methyltransferase inhibitors (14, 15), as we instead observe a prominent role for KZNFs in our system. Further studies will be required to test the functional consequences of upregulating hundreds of noncoding RNAs via oncogenic RAS signaling, as well as their potential utility as tissue-specific biomarkers of RAS-driven cancers.

One or more features from any embodiments described herein or in the figures may be combined with one or more features of any other embodiment described herein in the figures without departing from the scope of the disclosure.

All publications, patents and patent applications cited in this specification are herein incorporated by reference as if each individual publication or patent application were specifically and individually indicated to be incorporated by reference. Although the foregoing disclosure has been described in some detail by way of illustration and example for purposes of clarity of understanding, it will be readily apparent to those of ordinary skill in the art in light of the teachings of this disclosure that certain changes and modifications may be made thereto without departing from the spirit or scope of the appended claims.

### REFERENCES

1. J. T. Lee, Epigenetic regulation by long noncoding RNAs. Science 338, 1435-1439 (2012).
2. M. Kellis et al., Defining functional DNA elements in the human genome. Proc Natl Acad Sci USA 111, 6131-6138 (2014).
3. E. S. Lander et al., Initial sequencing and analysis of the human genome. Nature 409, 860-921 (2001).
4. K. H. Burns, Transposable elements in cancer. Nat Rev Cancer 17, 415-424 (2017).
5. G. Bourque et al., Ten things you should know about transposable elements. Genome Biol 19, 199 (2018).
6. E. Anastasiadou, L. S. Jacob, F. J. Slack, Non-coding RNA networks in cancer. Nat Rev Cancer 18, 5-18 (2018).
7. J. R. Evans, F. Y. Feng, A. M. Chinnaiyan, The bright side of dark matter: lncRNAs in cancer. J Clin Invest 126, 2775-2782 (2016).
8. D. H. Kim et al., Single-cell transcriptome analysis reveals dynamic changes in lncRNA expression during reprogramming. Cell Stem Cell 16, 88-101 (2015).
9. B. Papke, C. J. Der, Drugging RAS: Know the enemy. Science 355, 1158-1163 (2017).
10. A. S. Lundberg et al., Immortalization and transformation of primary human airway epithelial cells by gene transfer. Oncogene 21, 4577-4586 (2002).
11. R. K. Powers, A. Goodspeed, H. Pielke-Lombardo, A. C. Tan, J. C. Costello, GSEA-InContext: identifying novel and common patterns in expression experiments. Bioinformatics 34, 1555-1564 (2018).
12. E. Kim et al., Systematic Functional Interrogation of Rare Cancer Variants Identifies Oncogenic Alleles. Cancer Discov 6, 714-726 (2016).
13. A. J. Minn, Interferons and the Immunogenic Effects of Cancer Therapy. Trends Immunol 36, 725-737 (2015).
14. K. B. Chiappinelli et al., Inhibiting DNA Methylation Causes an Interferon Response in Cancer via dsRNA Including Endogenous Retroviruses. Cell 162, 974-986 (2015).
15. D. Roulois et al., DNA-Demethylating Agents Target Colorectal Cancer Cells by Inducing Viral Mimicry by Endogenous Transcripts. Cell 162, 961-973 (2015).
16. J. L. Benci et al., Opposing Functions of Interferon Coordinate Adaptive and Innate Immune Responses to Cancer Immune Checkpoint Blockade. Cell 178, 933-948 e914 (2019).
17. M. Imbeault, P. Y. Helleboid, D. Trono, KRAB zinc-finger proteins contribute to the evolution of gene regulatory networks. Nature 543, 550-554 (2017).
18. Bolger, A. M., Lohse, M., & Usadel, B. (2014). Trimmomatic: A flexible trimmer for Illumina Sequence Data. Bioinformatics, btu170.
19. Andrews S. (2010). FastQC: a quality control tool for high throughput sequence data.
20. Smit, A F A, Hubley, R & Green, P. RepeatMasker Open-4.0. 2013-2015
21. Patro, R., Duggal, G., Love, M. I., Irizarry, R. A. & Kingsford, C. Salmon provides fast and bias-aware quantification of transcript expression. Nature Methods 14, 417 (2017).
22. Harold J. Pimentel, Nicolas Bray, Suzette Puente, Páll Melsted and Lior Pachter, Differential analysis of RNA-Seq incorporating quantification uncertainty, Nature Methods (2017), advanced access.
23. Love, M. I., Huber, W., Anders, S. Moderated estimation of fold change and dispersion for RNA-seq data with DESeq2 Genome Biology 15(12):550 (2014).
24. Charlotte Soneson, Michael I. Love, Mark D. Robinson (2015): Differential analyses for RNA-seq: transcript-level estimates improve gene-level inferences.

F1000Research.
25. Guo, C., Jeong, H.-H., Hsieh, Y.-C., Klein, H.-U., Bennett, D. A., Jager, P. L. D., Liu, Z., and Shulman, J. M. (2018). Tau Activates Transposable Elements in Alzheimer's Disease. Cell Reports 23, 2874-2880.
26. R Core Team (2018). R: A language and environment for statistical computing. R Foundation for Statistical Computing, Vienna, Austria.
27. Hadley Wickham (2017). tidyverse: Easily Install and Load the ‘Tidyverse’. R package version 1.2.1. 28. Sergushichev A (2016). “An algorithm for fast preranked gene set enrichment analysis using cumulative statistic calculation.” bioRxiv. doi: 10.1101/060012.
29. Liberzon et al. 2011 Bioinformatics 27(12):1739-40.
30. Ashburner et al. Gene ontology: tool for the unification of biology (2000) Nat Genet 25(1):25-9. Online at Nature Genetics.
31. GO Consortium, Nucleic Acids Res., 2017.
32. Mi et al., Nucleic Acids Res., 2017.
33. Jennifer Bryan (2016). cellranger: Translate Spreadsheet Cell Ranges to Rows and Columns. R package version 1.1.0.
34. Stuart and Butler et al. Comprehensive integration of single cell data. bioRxiv (2018).

1. A method for diagnosing and/or treating cancer in a subject, the method comprising:
analyzing the expression level of one or more genes in Tables 1-3 in a biological sample from the subject in conjunction with a corresponding reference level for the gene in a control sample from a control subject,
wherein a differential expression level of the one or more genes in the biological sample from the subject compared to the corresponding reference level for the gene in the control sample from the control subject indicates that the subject has cancer.

2. The method of claim 1, wherein the cancer comprises a KRAS mutation.

3. The method of claim 2, wherein the KRAS mutation is in a tissue of the subject.

4. The method of claim 3, where the tissue is lung.

5. The method of claim 1, wherein the cancer is lung cancer.

6. The method of claim 5, wherein the lung cancer is lung adenocarcinoma.

7. The method of claim 1, further comprising, prior to analyzing, measuring the expression level of the one or more genes in Tables 1-3 and the expression level of the corresponding reference level for the gene in the control sample.

8. The method of claim 1, further comprising, after analyzing, administering to the subject one or more anticancer agents.

9. The method of claim 8, wherein the anticancer agent is an inhibitor of a K-ras gene.

10. The method of claim 8, wherein the anticancer agent is an inhibitor of the gene that is identified to have the differential expression level compared to the corresponding reference level for the gene in the control sample.

11. The method of claim 1, wherein the method comprises analyzing the expression level of a gene involved in the interferon (IFN) alpha or gamma response.

12. The method of claim 11, wherein an increase in the expression level of the gene involved in the IFN alpha or gamma response relative to a corresponding reference level for the gene in the control sample from the control subject indicates that the subject has cancer.

13. The method of claim 1, wherein the method comprises analyzing the expression level of a gene encoding a pattern recognition receptor (PRR).

14. The method of claim 13, wherein an increase in the expression level of the gene encoding the PRR relative to a corresponding reference level for the gene in the control sample from the control subject indicates that the subject has cancer.

15. The method of claim 1, wherein the method comprises analyzing the expression level of a gene encoding cytosolic RNA sensor RIG-I or MDA5.

16. The method of claim 15, wherein an increase in the expression level of the gene encoding cytosolic RNA sensor RIG-I or MDA5 relative to a corresponding reference level for the gene in the control sample from the control subject indicates that the subject has cancer.

17. The method of claim 1, wherein the method comprises analyzing the expression level of a gene encoding a KRAB zinc-finger (KZNF) protein.

18. The method of claim 17, wherein a decrease in the expression level of the gene encoding the KZNF protein relative to a corresponding reference level for the gene in the control sample from the control subject indicates that the subject has cancer.

19. The method of claim 7, wherein measuring the expression level of the one or more genes comprises performing polymerase chain reaction (PCR), reverse transcriptase polymerase chain reaction (RT-PCR), single-cell RNA-sequencing, microarray analysis, a Northern blot, serial analysis of gene expression (SAGE), immunoassay, hybridization capture, cDNA sequencing, direct RNA sequencing, nanopore sequencing, and/or mass spectrometry.

20. (canceled)

21. (canceled)

22. The method of claim 1, wherein the biological sample is a blood sample, a urine sample, or a tissue sample.

23.-26. (canceled)
